(19) United States
(12) Patent Application Publication
(10) Pub. No.: US 2026/0241025 A1
(43) Pub. Date: Aug. 20, 2026

(54) TREATMENT OF METASTATIC BRAIN TUMOR BY ADMINISTRATION OF ANTIBODY-DRUG CONJUGATE

(71) Applicant: DAIICHI SANKYO COMPANY, LIMITED, Tokyo (JP)

(72) Inventors: Kiyoshi SUGIHARA, Tokyo (JP); Chiaki ISHII, Tokyo (JP)

(73) Assignee: DAIICHI SANKYO COMPANY, LIMITED, Tokyo (JP)

(21) Appl. No.: 19/001,254

(22) Filed: Dec. 24, 2024

Related U.S. Application Data
(63) Continuation of application No. 17/264,250, filed on Jul. 30, 2019, now Pat. No. 12,220,604.

(30) Foreign Application Priority Data
Jul. 31, 2018 (JP) 2018-143372

(51) Int. Cl. A61K 47/68; A61K 31/4745; A61K 47/54; A61P 35/00; A61P 35/04; C07K 16/18; C07K 16/28; C07K 16/30; C07K 16/32

(52) U.S. Cl. CPC A61K 47/6851; A61K 31/4745; A61K 47/545; A61K 47/68037; A61K 47/6843; A61K 47/6849; A61K 47/6855; A61K 47/6889; A61P 35/00; A61P 35/04; C07K 16/18; C07K 16/28; C07K 16/2827; C07K 16/30; C07K 16/32

(57) ABSTRACT

A therapeutic agent for a metastatic brain tumor comprising, as an active component, an antibody-drug conjugate in which a drug-linker represented by the following formula (wherein A represents a connecting position to an antibody) is conjugated to the antibody via a thioether bond and/or a method of treatment for a metastatic brain tumor, comprising administering the antibody-drug conjugate to a subject in need of the treatment for a metastatic brain tumor.

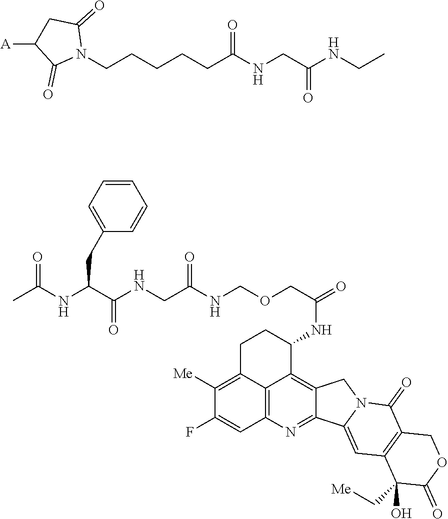

### CROSS-REFERENCE TO RELATED APPLICATIONS

The present application is a Continuation of U.S. patent application Ser. No. 17/264,250, filed on Jan. 28, 2021, which claims priority under 35 U.S.C. § 371 to International Patent Application No. PCT/JP2019/029765, filed Jul. 30, 2019, which claims priority to and the benefit of Japanese Patent Application No. 2018-143372, filed on Jul. 31, 2018. The contents of these applications are hereby incorporated by reference in their entireties.

### SEQUENCE LISTING

The instant application contains a Sequence Listing which has been submitted in XML format and is hereby incorporated by reference in its entirety. Said XML copy is named 122622-0200. xml, was created on Mar. 11, 2025, and is 20, 330 bytes in size.

### TECHNICAL FIELD

The present invention relates to a therapeutic agent for a metastatic brain tumor comprising a specific antibody-drug conjugate, and/or a method of treatment for a metastatic brain tumor comprising administering a specific antibody-drug conjugate to a subject.

### BACKGROUND ART

A metastatic brain tumor is a disease which develops when a primary cancer metastasizes to the brain and is known to occur in 20 to 40% of cancer patients. Cancer metastasis to the brain not only has a significant impact on prognosis but also leads to notable decline in QOL, and so the development of an effective method of treatment for a metastatic brain tumor is required (Non Patent References 1, 2).

As the primary cancer for metastatic brain tumors, lung cancer, breast cancer, melanoma, renal cell cancer, kidney cancer, colorectal cancer, gastric cancer, head and neck cancer, hepatocellular cancer, liver cancer, ovarian cancer, prostate cancer, bladder cancer, pancreatic cancer, endometrial cancer, thyroid cancer, malignant lymphoma and sarcoma can be exemplified. Among these, it is known that particularly metastatic brain tumors originated from lung cancer, breast cancer and melanoma as primary cancers are present at high percentages (Non Patent References 1, 2).

For the treatment of metastatic brain tumors originated from lung cancer as a primary cancer, clinical studies using gefitinib (Non Patent Reference 3), erlotinib (Non Patent Reference 4), afatinib (Non Patent Reference 5) and osimertinib (Non Patent Reference 6), which are epidermal growth factor receptor (EGFR) tyrosine kinase inhibitors, and crizotinib (Non Patent Reference 7), which is an ALK inhibitor, or the like, are known.

For the treatment of metastatic brain tumors originated from breast cancer as a primary cancer, clinical studies using trastuzumab, which is an anti-HER2 antibody (Non Patent Reference 8), and lapatinib, which is a human epidermal growth factor receptor 2 (HER2) tyrosine kinase inhibitor (Non Patent Reference 9), or the like, are known.

For the treatment of metastatic brain tumors originated from melanoma as a primary cancer, clinical studies using vemurafenib, which is a BRAF inhibitor (Non Patent Reference 10), or the like, are known.

However, these drug therapies have not been established as the standard therapies, and radiotherapy and surgical therapy are considered as the first choice.

An antibody-drug conjugate (ADC) having a drug with cytotoxicity conjugated to an antibody capable of binding to an antigen expressed on the surface of cancer cells and cellular internalization, can deliver the drug selectively to cancer cells and can thus be expected to cause accumulation of the drug within cancer cells and to kill the cancer cells (Non-Patent References 11 to 15).

As one such antibody-drug conjugate, an antibody-drug conjugate comprising an antibody and a derivative of exatecan, which is a topoisomerase I inhibitor, as its components is known (Patent References 1 to 5, Non Patent References 16 to 19). These antibody-drug conjugates, which exert a superior antitumor effect and safety, are currently under clinical studies.

For the treatment of metastatic brain tumors using an antibody-drug conjugate, nonclinical and clinical studies of the treatment of metastatic brain tumors originated from breast cancer as a primary cancer using trastuzumab emtansine, which is an anti-HER2 antibody-drug conjugate having, as a component, DM1, which is a tubulin inhibitor, are known (Non Patent References 20 to 23).

### CITATION LIST

### Patent Literature

Patent Reference 1: International Publication No. WO 2014/057687
Patent Reference 2: International Publication No. WO 2015/098099
Patent Reference 3: International Publication No. WO 2015/115091
Patent Reference 4: International Publication No. WO 2015/155998
Patent Reference 5: International Publication No. WO 2018/066626

### Non Patent Literature

Non Patent Reference 1: Lorenzo R., et al., Ther Adv Med Oncol. 2017 December; 9 (12): 781-796.
Non Patent Reference 2: Rahmathulla G., et al., Journal of Oncology 2012, Article ID 723541.
Non Patent Reference 3: Kim J-E., et al., Lung Cancer 2009; 65:351-354.
Non Patent Reference 4: Iuchi T., et al., Cancer 2013; 82:282-287.
Non Patent Reference 5: Hoffknecht P., et al., J. Thorac. Oncol. 2015; 10:156-163.
Non Patent Reference 6: Ahn MJ., et al., Eur. J. Cancer 2015; 51: S625-S626.
Non Patent Reference 7: Costa DB., et al., J. Clin. Oncol. 2015; 33:1881-1888.
Non Patent Reference 8: Bartsch R., et al., J. Neurooncol. 2007; 85:311-317.
Non Patent Reference 9: Lin NU., et al., Clin. Cancer Res. 2009; 15:1452-1459.
Non Patent Reference 10: Dummer R., et al., Eur. J. Cancer 2014; 50:611-621.
Non Patent Reference 11: Ducry L., et al., Bioconjugate Chem. (2010) 21, 5-13.
Non Patent Reference 12: Alley S. C., et al., Current Opinion in Chemical Biology (2010) 14, 529-537.
Non Patent Reference 13: Damle N. K., Expert Opin. Biol. Ther. (2004) 4, 1445-1452.
Non Patent Reference 14: Senter P. D., et al., Nature Biotechnology (2012) 30, 631-637.
Non Patent Reference 15: Howard A., et al., J Clin Oncol 29:398-405.
Non Patent Reference 16: Ogitani Y., et al., Clinical Cancer Research (2016) 22 (20), 5097-5108.
Non Patent Reference 17: Ogitani Y., et al., Cancer Science (2016) 107, 1039-1046.
Non Patent Reference 18: Doi T., et al., Lancet Oncol 2017; 18:1512-22.
Non Patent Reference 19: Takegawa N., et al., Int. J. Cancer: 141, 1682-1689 (2017)
Non Patent Reference 20: Bartsch R., et al., Clin. Exp. Metastasis (2015) 32:729-737.
Non Patent Reference 21: Askoxylakis V., et al., J. Natl. Cancer Inst. (2016) 108 (2): djv313.
Non Patent Reference 22: Ricciardi GRR., et al., BMC Cancer (2018) 18:97.
Non Patent Reference 23: Okines A., et al., Breast J. 2018; 24:253-259.

### SUMMARY OF INVENTION

### Technical Problem

Low molecular weight compounds and antibodies are used as cancer therapeutic agents but it is usually difficult for these to pass through the blood-brain barrier (BBB). Particularly, since antibodies have extremely high molecular weight, they cannot be substantially expected to pass through the blood-brain barrier (Bendell J C., et al., Cancer 2003; 97 (12): 2972-7). On the other hand, there is a report that an antibody intravenously administered for the purpose of treating a metastatic brain tumor passed through the Blood-brain barrier (Tamura K., et al., J. Nucl. Med. 2013; 54 (11): 1869-75). The reason therefor is inferred to be that the blood-brain barrier had been destroyed by the tumor that had metastasized to the brain, and thus the ability of the antibody to migrate to the brain relatively increased.

However, there is also a report that even in the case where intracerebral concentrations of small molecule compound and antibody showed considerable values, an expected therapeutic effect was not obtained. The reason therefor is inferred to be that the tumor that had metastasized to the brain had acquired drug resistance (for example, a gene mutation affecting downstream signaling and drug elimination mechanisms, or the like, such as P glycoprotein) (Saunus J M., et al., J. Pathol. 2015; 237:363-78, Brastianos P K., et al., Cancer Discov. 2015; 5:1164-77).

Further, it is also known that a blood-tumor barrier (BTB) is formed between a tumor metastasized to the brain and normal tissues in the brain, whereby drug resistance appears (Quail D F. et al., Cancer Cell 2017; 31:326-41).

An object of the present invention is to verify whether or not a specific antibody-drug conjugate including a derivative of exatecan as a component passes through the blood-brain barrier and whether or not it exerts an intended antitumor effect on a metastatic brain tumor which has acquired drug resistance. Additionally, another object is to provide a therapeutic agent for a metastatic brain tumor comprising the antibody-drug conjugate, and/or a method of treatment for a metastatic brain tumor comprising administering the antibody-drug conjugate to a subject.

### Solution to Problem

As a result of diligent studies in order to solve the above problems, the present inventors have found that a specific antibody-drug conjugate including a derivative of exatecan as a component exhibits an excellent antitumor effect on metastatic brain tumors, thereby accomplishing the present invention.

Thus, the present invention provides the following [1] to.

A therapeutic agent for a metastatic brain tumor comprising, as an active component, an antibody-drug conjugate in which a drug-linker represented by the following formula:

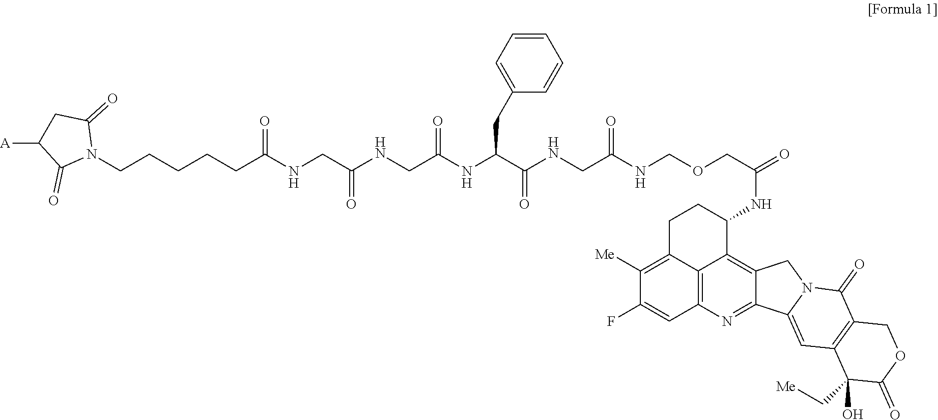

wherein A represents a connecting position to an antibody;
   is conjugated to the antibody via a thioether bond.

The therapeutic agent according to [1], wherein the primary cancer for the metastatic brain tumor is at least one selected from the group consisting of breast cancer, lung cancer, melanoma, renal cell cancer, kidney cancer, colorectal cancer, gastric cancer, head and neck cancer, hepatocellular cancer, liver cancer, ovarian cancer, prostate cancer, bladder cancer, pancreatic cancer, endometrial cancer, thyroid cancer, malignant lymphoma and sarcoma.

The therapeutic agent according to [1], wherein the primary cancer for the metastatic brain tumor is at least one selected from the group consisting of breast cancer, lung cancer and melanoma.

The therapeutic agent according to [1], wherein the primary cancer for the metastatic brain tumor is breast cancer.

The therapeutic agent according to [1], wherein the primary cancer for the metastatic brain tumor is lung cancer.

The therapeutic agent according to [1], wherein the primary cancer for the metastatic brain tumor is melanoma.

The therapeutic agent according to any one of [1] to [6], wherein the antibody in the antibody-drug conjugate is an anti-HER2 antibody, an anti-HER3 antibody, an anti-TROP2 antibody, an anti-B7-H3 antibody, an anti-GPR20 antibody, or an anti-CDH6 antibody.

The therapeutic agent according to [7], wherein the antibody in the antibody-drug conjugate is an anti-HER2 antibody.

The therapeutic agent according to [8], wherein the anti-HER2 antibody is an antibody comprising a heavy chain consisting of an amino acid sequence consisting of amino acid residues 1 to 449 of SEQ ID NO: 1 and a light chain consisting of an amino acid sequence consisting of amino acid residues 1 to 214 of SEQ ID NO: 2.

The therapeutic agent according to [8], wherein the anti-HER2 antibody is an antibody comprising a heavy chain consisting of an amino acid sequence represented by SEQ ID NO: 1 and a light chain consisting of an amino acid sequence represented by SEQ ID NO: 2.

The therapeutic agent according to any one of [8] to [10], wherein the average number of units of the drug-linker conjugated per antibody molecule in the antibody-drug conjugate is in the range of from 7 to 8.

The therapeutic agent according to [7], wherein the antibody in the antibody-drug conjugate is an anti-HER3 antibody.

The therapeutic agent according to [12], wherein the anti-HER3 antibody is an antibody comprising a heavy chain consisting of an amino acid sequence represented by SEQ ID NO: 3 and a light chain consisting of an amino acid sequence represented by SEQ ID NO: 4.

The therapeutic agent according to [13], wherein the anti-HER3 antibody lacks a lysine residue at the carboxyl terminus of the heavy chain.

The therapeutic agent according to any one of [12] to [14], wherein the average number of units of the drug-linker conjugated per antibody molecule in the antibody-drug conjugate is in the range of from 7 to 8.

The therapeutic agent according to [7], wherein the antibody in the antibody-drug conjugate is an anti-TROP2 antibody.

The therapeutic agent according to [16], wherein the anti-TROP2 antibody is an antibody comprising a heavy chain consisting of an amino acid sequence consisting of amino acid residues 20 to 470 of SEQ ID NO: 5 and a light chain consisting of an amino acid sequence consisting of amino acid residues 21 to 234 of SEQ ID NO: 6.

The therapeutic agent according to [17], wherein the anti-TROP2 antibody lacks a lysine residue at the carboxyl terminus of the heavy chain.

The therapeutic agent according to any one of [16] to [18], wherein the average number of units of the drug-linker conjugated per antibody molecule in the antibody-drug conjugate is in the range of from 3.5 to 4.5.

The therapeutic agent according to [7], wherein the antibody in the antibody-drug conjugate is an anti-B7-H3 antibody.

The therapeutic agent according to [20], wherein the anti-B7-H3 antibody is an antibody comprising a heavy chain consisting of an amino acid sequence consisting of amino acid residues 20 to 471 of SEQ ID NO: 7 and a light chain consisting of an amino acid sequence consisting of amino acid residues 21 to 233 of SEQ ID NO: 8.

The therapeutic agent according to [21], wherein the anti-B7-H3 antibody lacks a lysine residue at the carboxyl terminus of the heavy chain.

The therapeutic agent according to any one of [20] to [22], wherein the average number of units of the drug-linker conjugated per antibody molecule in the antibody-drug conjugate is in the range of from 3.5 to 4.5.

The therapeutic agent according to [7], wherein the antibody in the antibody-drug conjugate is an anti-GPR20 antibody.

The therapeutic agent according to [24], wherein the anti-GPR20 antibody is an antibody comprising a heavy chain consisting of an amino acid sequence consisting of amino acid residues 20 to 472 of SEQ ID NO: 9 and a light chain consisting of an amino acid sequence consisting of amino acid residues 21 to 234 of SEQ ID NO: 10.

The therapeutic agent according to [25], wherein the anti-GPR20 antibody lacks a lysine residue at the carboxyl terminus of the heavy chain.

The therapeutic agent according to any one of [24] to [26], wherein the average number of units of the drug-linker conjugated per antibody molecule in the antibody-drug conjugate is in the range of from 7 to 8.

The therapeutic agent according to [7], wherein the antibody in the antibody-drug conjugate is an anti-CDH6 antibody.

The therapeutic agent according to [28], wherein the anti-CDH6 antibody is an antibody comprising a heavy chain consisting of an amino acid sequence consisting of amino acid residues 20 to 471 of SEQ ID NO: 11 and a light chain consisting of an amino acid sequence consisting of amino acid residues 21 to 233 of SEQ ID NO: 12.

The therapeutic agent according to [29], wherein the anti-CDH6 antibody lacks a lysine residue at the carboxyl terminus of the heavy chain.

The therapeutic agent according to any one of [28] to [30], wherein the average number of units of the drug-linker conjugated per antibody molecule in the antibody-drug conjugate is in the range of from 7 to 8.

A therapeutic agent for a metastatic brain tumor comprising, as an active component, an antibody-drug conjugate represented by the following formula:

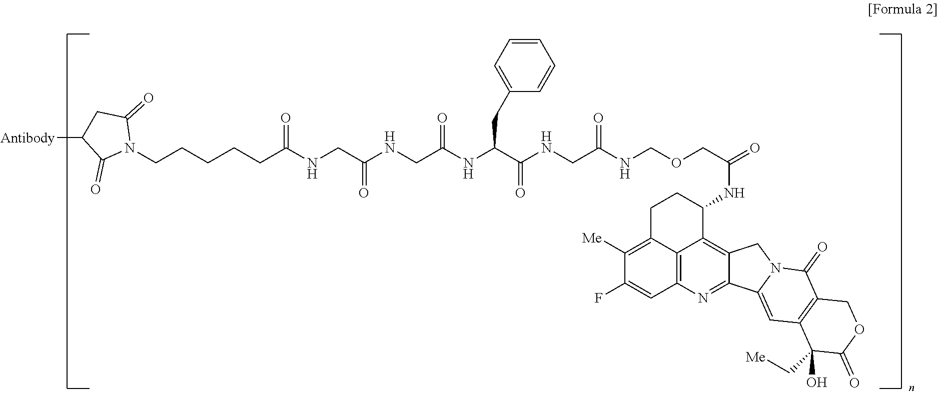

wherein the drug-linker is conjugated to the antibody via a thioether bond, and n is the average number of units of the drug-linker conjugated per antibody molecule.

The therapeutic agent according to [32], wherein the primary cancer for the metastatic brain tumor is at least one selected from the group consisting of breast cancer, lung cancer, melanoma, renal cell cancer, kidney cancer, colorectal cancer, gastric cancer, head and neck cancer, hepatocellular cancer, liver cancer, ovarian cancer, prostate cancer, bladder cancer, pancreatic cancer, endometrial cancer, thyroid cancer, malignant lymphoma and sarcoma.

The therapeutic agent according to [32], wherein the primary cancer for the metastatic brain tumor is at least one selected from the group consisting of breast cancer, lung cancer and melanoma.

The therapeutic agent according to [32], wherein the primary cancer for the metastatic brain tumor is breast cancer.

The therapeutic agent according to [32], wherein the primary cancer for the metastatic brain tumor is lung cancer.

The therapeutic agent according to [32], wherein the primary cancer for the metastatic brain tumor is melanoma.

The therapeutic agent according to any one of [32] to [37], wherein the antibody in the antibody-drug conjugate is an anti-HER2 antibody, an anti-HER3 antibody, an anti-TROP2 antibody, an anti-B7-H3 antibody, an anti-GPR20 antibody, or an anti-CDH6 antibody.

The therapeutic agent according to [38], wherein the antibody in the antibody-drug conjugate is an anti-HER2 antibody.

The therapeutic agent according to [39], wherein the anti-HER2 antibody is an antibody comprising a heavy chain consisting of an amino acid sequence consisting of amino acid residues 1 to 449 of SEQ ID NO: 1 and a light chain consisting of an amino acid sequence consisting of amino acid residues 1 to 214 of SEQ ID NO: 2.

The therapeutic agent according to [39], wherein the anti-HER2 antibody is an antibody comprising a heavy chain consisting of an amino acid sequence represented by SEQ ID NO: 1 and a light chain consisting of an amino acid sequence represented by SEQ ID NO: 2.

The therapeutic agent according to any one of [39] to [41], wherein the average number of units of the drug-linker conjugated per antibody molecule in the antibody-drug conjugate is in the range of from 7 to 8.

The therapeutic agent according to [38], wherein the antibody in the antibody-drug conjugate is an anti-HER3 antibody.

The therapeutic agent according to [43], wherein the anti-HER3 antibody is an antibody comprising a heavy chain consisting of an amino acid sequence represented by SEQ ID NO: 3 and a light chain consisting of an amino acid sequence represented by SEQ ID NO: 4.

The therapeutic agent according to [44], wherein the anti-HER3 antibody lacks a lysine residue at the carboxyl terminus of the heavy chain.

The therapeutic agent according to any one of [43] to [45], wherein the average number of units of the drug-linker conjugated per antibody molecule in the antibody-drug conjugate is in the range of from 7 to 8.

The therapeutic agent according to [38], wherein the antibody in the antibody-drug conjugate is an anti-TROP2 antibody.

The therapeutic agent according to [47], wherein the anti-TROP2 antibody is an antibody comprising a heavy chain consisting of an amino acid sequence consisting of amino acid residues 20 to 470 of SEQ ID NO: 5 and a light chain consisting of an amino acid sequence consisting of amino acid residues 21 to 234 of SEQ ID NO: 6.

The therapeutic agent according to [48], wherein the anti-TROP2 antibody lacks a lysine residue at the carboxyl terminus of the heavy chain.

The therapeutic agent according to any one of 47 to 49, wherein the average number of units of the drug-linker conjugated per antibody molecule in the antibody-drug conjugate is in the range of from 3.5 to 4.5.

The therapeutic agent according to [38], wherein the antibody in the antibody-drug conjugate is an anti-B7-H3 antibody.

The therapeutic agent according to [51], wherein the anti-B7-H3 antibody is an antibody comprising a heavy chain consisting of an amino acid sequence consisting of amino acid residues 20 to 471 of SEQ ID NO: 7 and a light chain consisting of an amino acid sequence consisting of amino acid residues 21 to 233 of SEQ ID NO: 8.

The therapeutic agent according to [52], wherein the anti-B7-H3 antibody lacks a lysine residue at the carboxyl terminus of the heavy chain.

The therapeutic agent according to any one of [51] to [53], wherein the average number of units of the drug-linker conjugated per antibody molecule in the antibody-drug conjugate is in the range of from 3.5 to 4.5.

The therapeutic agent according to [38], wherein the antibody in the antibody-drug conjugate is an anti-GPR20 antibody.

The therapeutic agent according to [55], wherein the anti-GPR20 antibody is an antibody comprising a heavy chain consisting of an amino acid sequence consisting of amino acid residues 20 to 472 of SEQ ID NO: 9 and a light chain consisting of an amino acid sequence consisting of amino acid residues 21 to 234 of SEQ ID NO: 10.

The therapeutic agent according to [56], wherein the anti-GPR20 antibody lacks a lysine residue at the carboxyl terminus of the heavy chain.

The therapeutic agent according to any one of [55] to [57], wherein the average number of units of the drug-linker conjugated per antibody molecule in the antibody-drug conjugate is in the range of from 7 to 8.

The therapeutic agent according to [38], wherein the antibody in the antibody-drug conjugate is an anti-CDH6 antibody.

[60] The therapeutic agent according to [59], wherein the anti-CDH6 antibody is an antibody comprising a heavy chain consisting of an amino acid sequence consisting of amino acid residues 20 to 471 of SEQ ID NO: 11 and a light chain consisting of an amino acid sequence consisting of amino acid residues 21 to 233 of SEQ ID NO: 12.

The therapeutic agent according to [60], wherein the anti-CDH6 antibody lacks a lysine residue at the carboxyl terminus of the heavy chain.

The therapeutic agent according to any one of [59] to [61], wherein the average number of units of the drug-linker conjugated per antibody molecule in the antibody-drug conjugate is in the range of from 7 to 8.

A method of treatment for a metastatic brain tumor, comprising administering an antibody-drug conjugate in which a drug-linker represented by the following formula:

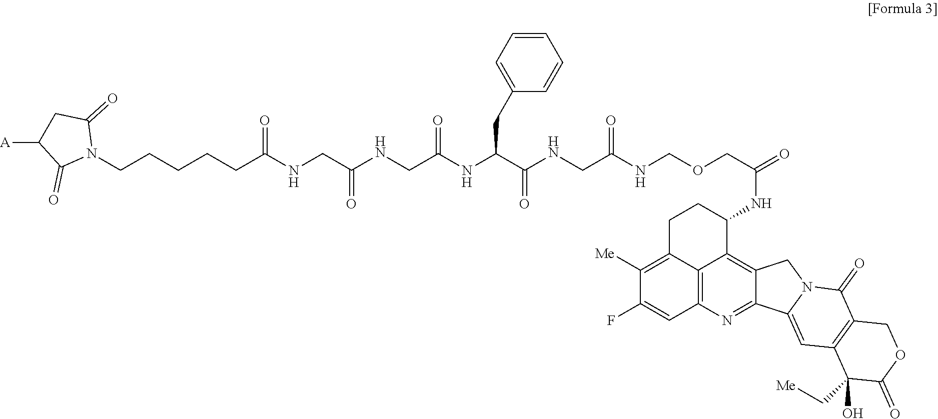

wherein A represents a connecting position to an antibody, is conjugated to the antibody via a thioether bond; to a subject in need of the treatment for a metastatic brain tumor.

The method of treatment according to [63], wherein the primary cancer for the metastatic brain tumor is at least one selected from the group consisting of breast cancer, lung cancer, melanoma, renal cell cancer, kidney cancer, colorectal cancer, gastric cancer, head and neck cancer, hepatocellular cancer, liver cancer, ovarian cancer, prostate cancer, bladder cancer, pancreatic cancer, endometrial cancer, thyroid cancer, malignant lymphoma and sarcoma.

The method of treatment according to [63], wherein the primary cancer for the metastatic brain tumor is at least one selected from the group consisting of breast cancer, lung cancer and melanoma.

The method of treatment according to [63], wherein the primary cancer for the metastatic brain tumor is breast cancer.

The method of treatment according to [63], wherein the primary cancer for the metastatic brain tumor is lung cancer.

The method of treatment according to [63], wherein the primary cancer for the metastatic brain tumor is melanoma.

The method of treatment according to any one of [63] to [68], wherein the antibody in the antibody-drug conjugate is an anti-HER2 antibody, an anti-HER3 antibody, an anti-TROP2 antibody, an anti-B7-H3 antibody, an anti-GPR20 antibody or an anti-CDH6 antibody.

The method of treatment according to [69], wherein the antibody in the antibody-drug conjugate is an anti-HER2 antibody.

The method of treatment according to [70], wherein the anti-HER2 antibody is an antibody comprising a heavy chain consisting of an amino acid sequence consisting of amino acid residues 1 to 449 of SEQ ID NO: 1 and a light chain consisting of an amino acid sequence consisting of amino acid residues 1 to 214 of SEQ ID NO: 2.

The method of treatment according to [70], wherein the anti-HER2 antibody is an antibody comprising a heavy chain consisting of an amino acid sequence represented by SEQ ID NO: 1 and a light chain consisting of an amino acid sequence represented by SEQ ID NO: 2.

The method of treatment according to any one of [70] to [72], wherein the average number of units of the drug-linker conjugated per antibody molecule in the antibody-drug conjugate is in the range of from 7 to 8.

The method of treatment according to [69], wherein the antibody in the antibody-drug conjugate is an anti-HER3 antibody.

The method of treatment according to [74], wherein the anti-HER3 antibody is an antibody comprising a heavy chain consisting of an amino acid sequence represented by SEQ ID NO: 3 and a light chain consisting of an amino acid sequence represented by SEQ ID NO: 4.

The method of treatment according to [75], wherein the anti-HER3 antibody lacks a lysine residue at the carboxyl terminus of the heavy chain.

The method of treatment according to any one of [74] to [76], wherein the average number of units of the drug-linker conjugated per antibody molecule in the antibody-drug conjugate is in the range of from 7 to 8.

The method of treatment according to [69], wherein the antibody in the antibody-drug conjugate is an anti-TROP2 antibody.

The method of treatment according to [78], wherein the anti-TROP2 antibody is an antibody comprising a heavy chain consisting of an amino acid sequence consisting of amino acid residues 20 to 470 of SEQ ID NO: 5 and a light chain consisting of an amino acid sequence consisting of amino acid residues 21 to 234 of SEQ ID NO: 6.

The method of treatment according to [79], wherein the anti-TROP2 antibody lacks a lysine residue at the carboxyl terminus of the heavy chain.

The method of treatment according to any one of [78] to [80], wherein the average number of units of the drug-linker conjugated per antibody molecule in the antibody-drug conjugate is in the range of from 3.5 to 4.5.

The method of treatment according to [69], wherein the antibody in the antibody-drug conjugate is an anti-B7-H3 antibody.

The method of treatment according to [82], wherein the anti-B7-H3 antibody is an antibody comprising a heavy chain consisting of an amino acid sequence consisting of amino acid residues 20 to 471 of SEQ ID NO: 7 and a light chain consisting of an amino acid sequence consisting of amino acid residues 21 to 233 of SEQ ID NO: 8.

The method of treatment according to [83], wherein the anti-B7-H3 antibody lacks a lysine residue at the carboxyl terminus of the heavy chain.

The method of treatment according to any one of [82] to [84], wherein the average number of units of the drug-linker conjugated per antibody molecule in the antibody-drug conjugate is in the range of from 3.5 to 4.5.

The method of treatment according to [69], wherein the antibody in the antibody-drug conjugate is an anti-GPR20 antibody.

The method of treatment according to [86], wherein the anti-GPR20 antibody is an antibody comprising a heavy chain consisting of an amino acid sequence consisting of amino acid residues 20 to 472 of SEQ ID NO: 9 and a light chain consisting of an amino acid sequence consisting of amino acid residues 21 to 234 of SEQ ID NO: 10.

The method of treatment according to [87], wherein the anti-GPR20 antibody lacks a lysine residue at the carboxyl terminus of the heavy chain.

The method of treatment according to any one of [86] to [88], wherein the average number of units of the drug-linker conjugated per antibody molecule in the antibody-drug conjugate is in the range of from 7 to 8.

The method of treatment according to [69], wherein the antibody in the antibody-drug conjugate is an anti-CDH6 antibody.

The method of treatment according to [90], wherein the anti-CDH6 antibody is an antibody comprising a heavy chain consisting of an amino acid sequence consisting of amino acid residues 20 to 471 of SEQ ID NO: 11 and a light chain consisting of an amino acid sequence consisting of amino acid residues 21 to 233 of SEQ ID NO: 12.

The method of treatment according to [91], wherein the anti-CDH6 antibody lacks a lysine residue at the carboxyl terminus of the heavy chain.

The method of treatment according to any one of [90] to [92], wherein the average number of units of the drug-linker conjugated per antibody molecule in the antibody-drug conjugate is in the range of from 7 to 8.

A method of treatment for a metastatic brain tumor, comprising administering an antibody-drug conjugate represented by the following formula:

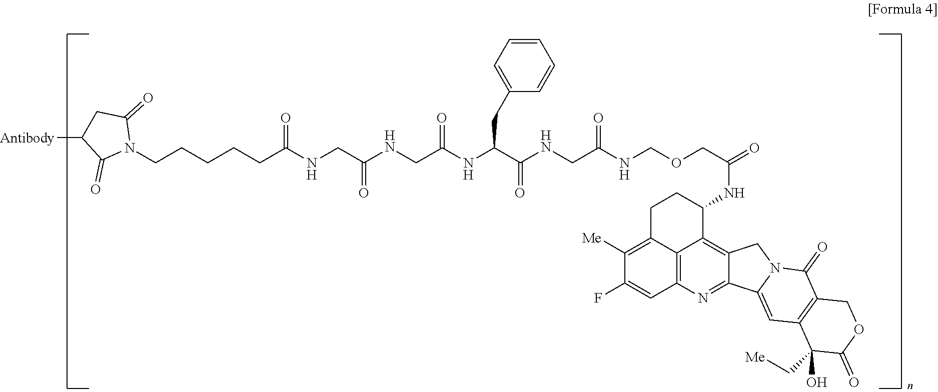

wherein the drug-linker is conjugated to the antibody via a thioether bond, and n is the average number of units of the drug-linker conjugated per antibody molecule;
to a subject in need of the treatment for a metastatic brain tumor.

The method of treatment according to [94], wherein the primary cancer for the metastatic brain tumor is at least one selected from the group consisting of breast cancer, lung cancer, melanoma, renal cell cancer, kidney cancer, colorectal cancer, gastric cancer, head and neck cancer, hepatocellular cancer, liver cancer, ovarian cancer, prostate cancer, bladder cancer, pancreatic cancer, endometrial cancer, thyroid cancer, malignant lymphoma and sarcoma.

The method of treatment according to [94], wherein the primary cancer for the metastatic brain tumor is at least one selected from the group consisting of breast cancer, lung cancer and melanoma.

The method of treatment according to [94], wherein the primary cancer for the metastatic brain tumor is breast cancer.

The method of treatment according to [94], wherein the primary cancer for the metastatic brain tumor is lung cancer.

The method of treatment according to [94], wherein the primary cancer for the metastatic brain tumor is melanoma.

The method of treatment according to any one of [94] to [99], wherein the antibody in the antibody-drug conjugate is an anti-HER2 antibody, an anti-HER3 antibody, an anti-TROP2 antibody, an anti-B7-H3 antibody, an anti-GPR20 antibody or an anti-CDH6 antibody.

The method of treatment according to [100], wherein the antibody in the antibody-drug conjugate is an anti-HER2 antibody.

The method of treatment according to [101], wherein the anti-HER2 antibody is an antibody comprising a heavy chain consisting of an amino acid sequence consisting of amino acid residues 1 to 449 of SEQ ID NO: 1 and a light chain consisting of an amino acid sequence consisting of amino acid residues 1 to 214 of SEQ ID NO: 2.

The method of treatment according to [101], wherein the anti-HER2 antibody is an antibody comprising a heavy chain consisting of an amino acid sequence represented by SEQ ID NO: 1 and a light chain consisting of an amino acid sequence represented by SEQ ID NO: 2.

The method of treatment according to any one of [101] to [103], wherein the average number of units of the drug-linker conjugated per antibody molecule in the antibody-drug conjugate is in the range of from 7 to 8.

The method of treatment according to [100], wherein the antibody in the antibody-drug conjugate is an anti-HER3 antibody.

The method of treatment according to [105], wherein the anti-HER3 antibody is an antibody comprising a heavy chain consisting of an amino acid sequence represented by SEQ ID NO: 3 and a light chain consisting of an amino acid sequence represented by SEQ ID NO: 4.

The method of treatment according to [106], wherein the anti-HER3 antibody lacks a lysine residue at the carboxyl terminus of the heavy chain.

The method of treatment according to any one of [105] to [107], wherein the average number of units of the drug-linker conjugated per antibody molecule in the antibody-drug conjugate is in the range of from 7 to 8.

The method of treatment according to [100], wherein the antibody in the antibody-drug conjugate is an anti-TROP2 antibody.

The method of treatment according to [109], wherein the anti-TROP2 antibody is an antibody comprising a heavy chain consisting of an amino acid sequence consisting of amino acid residues 20 to 470 of SEQ ID NO: 5 and a light chain consisting of an amino acid sequence consisting of amino acid residues 21 to 234 of SEQ ID NO: 6.

The method of treatment according to [110], wherein the anti-TROP2 antibody lacks a lysine residue at the carboxyl terminus of the heavy chain.

The method of treatment according to any one of [109] to [111] wherein the average number of units of the drug-linker conjugated per antibody molecule in the antibody-drug conjugate is in the range of from 3.5 to 4.5.

The method of treatment according to [100], wherein the antibody in the antibody-drug conjugate is an anti-B7-H3 antibody.

The method of treatment according to [113], wherein the anti-B7-H3 antibody is an antibody comprising a heavy chain consisting of an amino acid sequence consisting of amino acid residues 20 to 471 of SEQ ID NO: 7 and a light chain consisting of an amino acid sequence consisting of amino acid residues 21 to 233 of SEQ ID NO: 8.

The method of treatment according to [114], wherein the anti-B7-H3 antibody lacks a lysine residue at the carboxyl terminus of the heavy chain.

The method of treatment according to any one of [113] to [115], wherein the average number of units of the drug-linker conjugated per antibody molecule in the antibody-drug conjugate is in the range of from 3.5 to 4.5.

The method of treatment according to [100], wherein the antibody in the antibody-drug conjugate is an anti-GPR20 antibody.

The method of treatment according to [117], wherein the anti-GPR20 antibody is an antibody comprising a heavy chain consisting of an amino acid sequence consisting of amino acid residues 20 to 472 of SEQ ID NO: 9 and a light chain consisting of an amino acid sequence consisting of amino acid residues 21 to 234 of SEQ ID NO: 10.

The method of treatment according to [118], wherein the anti-GPR20 antibody lacks a lysine residue at the carboxyl terminus of the heavy chain.

The method of treatment according to any one of [117] to [119], wherein the average number of units of the drug-linker conjugated per antibody molecule in the antibody-drug conjugate is in the range of from 7 to 8.

The method of treatment according to [100], wherein the antibody in the antibody-drug conjugate is an anti-CDH6 antibody.

The method of treatment according to [121], wherein the anti-CDH6 antibody is an antibody comprising a heavy chain consisting of an amino acid sequence consisting of amino acid residues 20 to 471 of SEQ ID NO: 11 and a light chain consisting of an amino acid sequence consisting of amino acid residues 21 to 233 of SEQ ID NO: 12.

The method of treatment according to [122], wherein the anti-CDH6 antibody lacks a lysine residue at the carboxyl terminus of the heavy chain.

The method of treatment according to any one of [121] to [123], wherein the average number of units of the drug-linker conjugated per antibody molecule in the antibody-drug conjugate is in the range of from 7 to 8.

An antibody-drug conjugate, for use in treating a metastatic brain tumor, in which a drug-linker represented by the following formula:

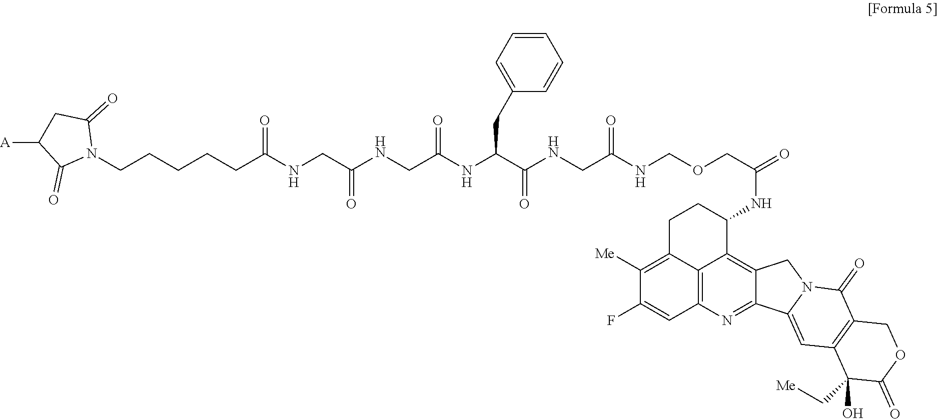

wherein A represents a connecting position to an antibody;
   is conjugated to the antibody via a thioether bond.

The antibody-drug conjugate according to [125], wherein the primary cancer for the metastatic brain tumor is at least one selected from the group consisting of breast cancer, lung cancer, melanoma, renal cell cancer, kidney cancer, colorectal cancer, gastric cancer, head and neck cancer, hepatocellular cancer, liver cancer, ovarian cancer, prostate cancer, bladder cancer, pancreatic cancer, endometrial cancer, thyroid cancer, malignant lymphoma and sarcoma.

The antibody-drug conjugate according to [125], wherein the primary cancer for the metastatic brain tumor is at least one selected from the group consisting of breast cancer, lung cancer and melanoma.

The antibody-drug conjugate according to [125], wherein the primary cancer for the metastatic brain tumor is breast cancer.

The antibody-drug conjugate according to [125], wherein the primary cancer for the metastatic brain tumor is lung cancer.

The antibody-drug conjugate according to [125], wherein the primary cancer for the metastatic brain tumor is melanoma.

The antibody-drug conjugate according to any one of [125] to [130], wherein the antibody in the antibody-drug conjugate is an anti-HER2 antibody, an anti-HER3 antibody, an anti-TROP2 antibody, an anti-B7-H3 antibody, an anti-GPR20 antibody or an anti-CDH6 antibody.

The antibody-drug conjugate according to [131], wherein the antibody in the antibody-drug conjugate is an anti-HER2 antibody

The antibody-drug conjugate according to [132], wherein the anti-HER2 antibody is an antibody comprising a heavy chain consisting of an amino acid sequence consisting of amino acid residues 1 to 449 of SEQ ID NO: 1 and a light chain consisting of an amino acid sequence consisting of amino acid residues 1 to 214 of SEQ ID NO: 2.

The antibody-drug conjugate according to [132], wherein the anti-HER2 antibody is an antibody comprising a heavy chain consisting of an amino acid sequence represented by SEQ ID NO: 1 and a light chain consisting of an amino acid sequence represented by SEQ ID NO: 2.

The antibody-drug conjugate according to any one of [132] to [134], wherein the average number of units of the drug-linker conjugated per antibody molecule in the antibody-drug conjugate is in the range of from 7 to 8.

The antibody-drug conjugate according to [131], wherein the antibody in the antibody-drug conjugate is an anti-HER3 antibody.

The antibody-drug conjugate according to [136], wherein the anti-HER3 antibody is an antibody comprising a heavy chain consisting of an amino acid sequence represented by SEQ ID NO: 3 and a light chain consisting of an amino acid sequence represented by SEQ ID NO: 4.

The antibody-drug conjugate according to [137], wherein the anti-HER3 antibody lacks a lysine residue at the carboxyl terminus of the heavy chain.

The antibody-drug conjugate according to any one of [136] to [138], wherein the average number of units of the drug-linker conjugated per antibody molecule in the antibody-drug conjugate is in the range of from 7 to 8.

The antibody-drug conjugate according to [131], wherein the antibody in the antibody-drug conjugate is an anti-TROP2 antibody.

The antibody-drug conjugate according to [140], wherein the anti-TROP2 antibody is an antibody comprising a heavy chain consisting of an amino acid sequence consisting of amino acid residues 20 to 470 of SEQ ID NO: 5 and a light chain consisting of an amino acid sequence consisting of amino acid residues 21 to 234 of SEQ ID NO: 6.

The antibody-drug conjugate according to [141], wherein the anti-TROP2 antibody lacks a lysine residue at the carboxyl terminus of the heavy chain.

The antibody-drug conjugate according to any one of [140] to [142], wherein the average number of units of the drug-linker conjugated per antibody molecule in the antibody-drug conjugate is in the range of from 3.5 to 4.5.

The antibody-drug conjugate according to [131], wherein the antibody in the antibody-drug conjugate is an anti-B7-H3 antibody.

The antibody-drug conjugate according to [144], wherein the anti-B7-H3 antibody is an antibody comprising a heavy chain consisting of an amino acid sequence consisting of amino acid residues 20 to 471 of SEQ ID NO: 7 and a light chain consisting of an amino acid sequence consisting of amino acid residues 21 to 233 of SEQ ID NO: 8.

The antibody-drug conjugate according to [145], wherein the anti-B7-H3 antibody lacks a lysine residue at the carboxyl terminus of the heavy chain.

The antibody-drug conjugate according to any one of [144] to [146], wherein the average number of units of the drug-linker conjugated per antibody molecule in the antibody-drug conjugate is in the range of from 3.5 to 4.5.

The antibody-drug conjugate according to [131], wherein the antibody in the antibody-drug conjugate is an anti-GPR20 antibody.

The antibody-drug conjugate according to [148], wherein the anti-GPR20 antibody is an antibody comprising a heavy chain consisting of an amino acid sequence consisting of amino acid residues 20 to 472 of SEQ ID NO: 9 and a light chain consisting of an amino acid sequence consisting of amino acid residues 21 to 234 of SEQ ID NO: 10.

The antibody-drug conjugate according to [149], wherein the anti-GPR20 antibody lacks a lysine residue at the carboxyl terminus of the heavy chain.

The antibody-drug conjugate according to any one of to [150], wherein the average number of units of the drug-linker conjugated per antibody molecule in the antibody-drug conjugate is in the range of from 7 to 8.

The antibody-drug conjugate according to [131], wherein the antibody in the antibody-drug conjugate is an anti-CDH6 antibody.

The antibody-drug conjugate according to [152], wherein the anti-CDH6 antibody is an antibody comprising a heavy chain consisting of an amino acid sequence consisting of amino acid residues 20 to 471 of SEQ ID NO: 11 and a light chain consisting of an amino acid sequence consisting of amino acid residues 21 to 233 of SEQ ID NO: 12.

The antibody-drug conjugate according to [153], wherein the anti-CDH6 antibody lacks a lysine residue at the carboxyl terminus of the heavy chain.

The antibody-drug conjugate according to any one of to [154], wherein the average number of units of the drug-linker conjugated per antibody molecule in the antibody-drug conjugate is in the range of from 7 to 8.

An antibody-drug conjugate, for use in treating a metastatic brain tumor, represented by the following formula:

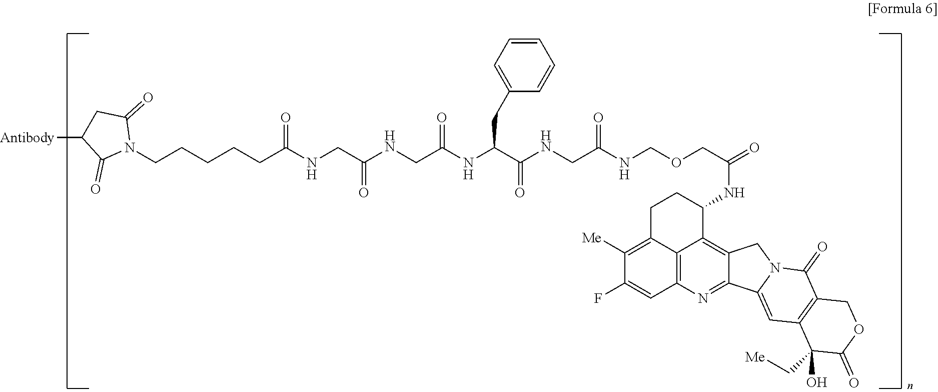

wherein the drug-linker is conjugated to the antibody via a thioether bond, and n is the average number of units of the drug-linker conjugated per antibody molecule.

The antibody-drug conjugate according to [156], wherein the primary cancer for the metastatic brain tumor is at least one selected from the group consisting of breast cancer, lung cancer, melanoma, renal cell cancer, kidney cancer, colorectal cancer, gastric cancer, head and neck cancer, hepatocellular cancer, liver cancer, ovarian cancer, prostate cancer, bladder cancer, pancreatic cancer, endometrial cancer, thyroid cancer, malignant lymphoma and sarcoma.

The antibody-drug conjugate according to [156], wherein the primary cancer for the metastatic brain tumor is at least one selected from the group consisting of breast cancer, lung cancer and melanoma.

The antibody-drug conjugate according to [156], wherein the primary cancer for the metastatic brain tumor is breast cancer.

The antibody-drug conjugate according to [156], wherein the primary cancer for the metastatic brain tumor is lung cancer.

The antibody-drug conjugate according to [156], wherein the primary cancer for the metastatic brain tumor is melanoma.

The antibody-drug conjugate according to any one of [156] to [161], wherein the antibody in the antibody-drug conjugate is an anti-HER2 antibody, an anti-HER3 antibody, an anti-TROP2 antibody, an anti-B7-H3 antibody, an anti-GPR20 antibody or an anti-CDH6 antibody.

The antibody-drug conjugate according to [162], wherein the antibody in the antibody-drug conjugate is an anti-HER2 antibody.

The antibody-drug conjugate according to [163], wherein the anti-HER2 antibody is an antibody comprising a heavy chain consisting of an amino acid sequence consisting of amino acid residues 1 to 449 of SEQ ID NO: 1 and a light chain consisting of an amino acid sequence consisting of amino acid residues 1 to 214 of SEQ ID NO: 2.

The antibody-drug conjugate according to [163], wherein the anti-HER2 antibody is an antibody comprising a heavy chain consisting of an amino acid sequence represented by SEQ ID NO: 1 and a light chain consisting of an amino acid sequence represented by SEQ ID NO: 2.

The antibody-drug conjugate according to any one of to [165], wherein the average number of units of the drug-linker conjugated per antibody molecule in the antibody-drug conjugate is in the range of from 7 to 8.

The antibody-drug conjugate according to [162], wherein the antibody in the antibody-drug conjugate is an anti-HER3 antibody.

The antibody-drug conjugate according to [167], wherein the anti-HER3 antibody is an antibody comprising a heavy chain consisting of an amino acid sequence represented by SEQ ID NO: 3 and a light chain consisting of an amino acid sequence represented by SEQ ID NO: 4.

The antibody-drug conjugate according to [168], wherein the anti-HER3 antibody lacks a lysine residue at the carboxyl terminus of the heavy chain.

The antibody-drug conjugate according to any one of [167] to [169], wherein the average number of units of the drug-linker conjugated per antibody molecule in the antibody-drug conjugate is in the range of from 7 to 8.

The antibody-drug conjugate according to [162], wherein the antibody in the antibody-drug conjugate is an anti-TROP2 antibody.

The antibody-drug conjugate according to [171], wherein the anti-TROP2 antibody is an antibody comprising a heavy chain consisting of an amino acid sequence consisting of amino acid residues 20 to 470 of SEQ ID NO: 5 and a light chain consisting of an amino acid sequence consisting of amino acid residues 21 to 234 of SEQ ID NO: 6.

The antibody-drug conjugate according to [172], wherein the anti-TROP2 antibody lacks a lysine residue at the carboxyl terminus of the heavy chain.

The antibody-drug conjugate according to any one of to [173], wherein the average number of units of the drug-linker conjugated per antibody molecule in the antibody-drug conjugate is in the range of from 3.5 to 4.5.

The antibody-drug conjugate according to [162], wherein the antibody in the antibody-drug conjugate is an anti-B7-H3 antibody.

The antibody-drug conjugate according to [175], wherein the anti-B7-H3 antibody is an antibody comprising a heavy chain consisting of an amino acid sequence consisting of amino acid residues 20 to 471 of SEQ ID NO: 7 and a light chain consisting of an amino acid sequence consisting of amino acid residues 21 to 233 of SEQ ID NO: 8.

The antibody-drug conjugate according to [176], wherein the anti-B7-H3 antibody lacks a lysine residue at the carboxyl terminus of the heavy chain.

The antibody-drug conjugate according to any one of [175] to [177], wherein the average number of units of the drug-linker conjugated per antibody molecule in the antibody-drug conjugate is in the range of from 3.5 to 4.5.

The antibody-drug conjugate according to [162], wherein the antibody in the antibody-drug conjugate is an anti-GPR20 antibody.

The antibody-drug conjugate according to [179], wherein the anti-GPR20 antibody is an antibody comprising a heavy chain consisting of an amino acid sequence consisting of amino acid residues 20 to 472 of SEQ ID NO: 9 and a light chain consisting of an amino acid sequence consisting of amino acid residues 21 to 234 of SEQ ID NO: 10.

The antibody-drug conjugate according to [180], wherein the anti-GPR20 antibody lacks a lysine residue at the carboxyl terminus of the heavy chain.

The antibody-drug conjugate according to any one of [179] to [181], wherein the average number of units of the drug-linker conjugated per antibody molecule in the antibody-drug conjugate is in the range of from 7 to 8.

The antibody-drug conjugate according to [162], wherein the antibody in the antibody-drug conjugate is an anti-CDH6 antibody.

The antibody-drug conjugate according to [183], wherein the anti-CDH6 antibody is an antibody comprising a heavy chain consisting of an amino acid sequence consisting of amino acid residues 20 to 471 of SEQ ID NO: 11 and a light chain consisting of an amino acid sequence consisting of amino acid residues 21 to 233 of SEQ ID NO: 12.

The antibody-drug conjugate according to [184], wherein the anti-CDH6 antibody lacks a lysine residue at the carboxyl terminus of the heavy chain.

The antibody-drug conjugate according to any one of to [185], wherein the average number of units of the drug-linker conjugated per antibody molecule in the antibody-drug conjugate is in the range of from 7 to 8.

Use of an antibody-drug conjugate for the manufacture of a medicament for treating a metastatic brain tumor, in which a drug-linker represented by the following formula:

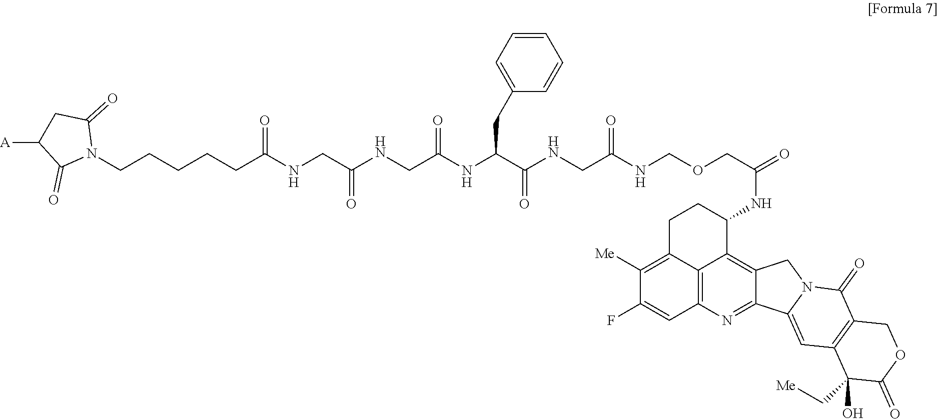

wherein A represents a connecting position to an antibody;
   is conjugated to the antibody via a thioether bond.

The use according to [187], wherein the primary cancer for the metastatic brain tumor is at least one selected from the group consisting of breast cancer, lung cancer, melanoma, renal cell cancer, kidney cancer, colorectal cancer, gastric cancer, head and neck cancer, hepatocellular cancer, liver cancer, ovarian cancer, prostate cancer, bladder cancer, pancreatic cancer, endometrial cancer, thyroid cancer, malignant lymphoma and sarcoma.

The use according to [187], wherein the primary cancer for the metastatic brain tumor is at least one selected from the group consisting of breast cancer, lung cancer and melanoma.

The use according to [187], wherein the primary cancer for the metastatic brain tumor is breast cancer.

The use according to [187], wherein the primary cancer for the metastatic brain tumor is lung cancer.

The use according to [187], wherein the primary cancer for the metastatic brain tumor is melanoma.

The use according to any one of [187] to [192], wherein the antibody in the antibody-drug conjugate is an anti-HER2 antibody, an anti-HER3 antibody, an anti-TROP2 antibody, an anti-B7-H3 antibody, an anti-GPR20 antibody or an anti-CDH6 antibody.

The use according to [193], wherein the antibody in the antibody-drug conjugate is an anti-HER2 antibody.

The use according to [194], wherein the anti-HER2 antibody is an antibody comprising a heavy chain consisting of an amino acid sequence consisting of amino acid residues 1 to 449 of SEQ ID NO: 1 and a light chain consisting of an amino acid sequence consisting of amino acid residues 1 to 214 of SEQ ID NO: 2.

The use according to [194], wherein the anti-HER2 antibody is an antibody comprising a heavy chain consisting of an amino acid sequence represented by SEQ ID NO: 1 and a light chain consisting of an amino acid sequence represented by SEQ ID NO: 2.

The use according to any one of [194] to [196], wherein the average number of units of the drug-linker conjugated per antibody molecule in the antibody-drug conjugate is in the range of from 7 to 8.

The use according to [193], wherein the antibody in the antibody-drug conjugate is an anti-HER3 antibody.

The use according to [198], wherein the anti-HER3 antibody is an antibody comprising a heavy chain consisting of an amino acid sequence represented by SEQ ID NO: 3 and a light chain consisting of an amino acid sequence represented by SEQ ID NO: 4.

The use according to [199], wherein the anti-HER3 antibody lacks a lysine residue at the carboxyl terminus of the heavy chain.

The use according to any one of [198] to [200], wherein the average number of units of the drug-linker conjugated per antibody molecule in the antibody-drug conjugate is in the range of from 7 to 8.

The use according to [193], wherein the antibody in the antibody-drug conjugate is an anti-TROP2 antibody.

The use according to [202], wherein the anti-TROP2 antibody is an antibody comprising a heavy chain consisting of an amino acid sequence consisting of amino acid residues 20 to 470 of SEQ ID NO: 5 and a light chain consisting of an amino acid sequence consisting of amino acid residues 21 to 234 of SEQ ID NO: 6.

The use according to [203], wherein the anti-TROP2 antibody lacks a lysine residue at the carboxyl terminus of the heavy chain.

The use according to any one of [202] to [204], wherein the average number of units of the drug-linker conjugated per antibody molecule in the antibody-drug conjugate is in the range of from 3.5 to 4.5.

The use according to [193], wherein the antibody in the antibody-drug conjugate is an anti-B7-H3 antibody.

The use according to [206], wherein the anti-B7-H3 antibody is an antibody comprising a heavy chain consisting of an amino acid sequence consisting of amino acid residues 20 to 471 of SEQ ID NO: 7 and a light chain consisting of an amino acid sequence consisting of amino acid residues 21 to 233 of SEQ ID NO: 8.

The use according to [207], wherein the anti-B7-H3 antibody lacks a lysine residue at the carboxyl terminus of the heavy chain.

The use according to any one of [206] to [208], wherein the average number of units of the drug-linker conjugated per antibody molecule in the antibody-drug conjugate is in the range of from 3.5 to 4.5.

The use according to [193], wherein the antibody in the antibody-drug conjugate is an anti-GPR20 antibody.

The use according to [210], wherein the anti-GPR20 antibody is an antibody comprising a heavy chain consisting of an amino acid sequence consisting of amino acid residues 20 to 472 of SEQ ID NO: 9 and a light chain consisting of an amino acid sequence consisting of amino acid residues 21 to 234 of SEQ ID NO: 10.

The use according to [211], wherein the anti-GPR20 antibody lacks a lysine residue at the carboxyl terminus of the heavy chain.

The use according to any one of [210] to [212], wherein the average number of units of the drug-linker conjugated per antibody molecule in the antibody-drug conjugate is in the range of from 7 to 8.

The use according to [193], wherein the antibody in the antibody-drug conjugate is an anti-CDH6 antibody.

The use according to [214], wherein the anti-CDH6 antibody is an antibody comprising a heavy chain consisting of an amino acid sequence consisting of amino acid residues 20 to 471 of SEQ ID NO: 11 and a light chain consisting of an amino acid sequence consisting of amino acid residues 21 to 233 of SEQ ID NO: 12.

The use according to [215], wherein the anti-CDH6 antibody lacks a lysine residue at the carboxyl terminus of the heavy chain.

The use according to any one of [214] to [216], wherein the average number of units of the drug-linker conjugated per antibody molecule in the antibody-drug conjugate is in the range of from 7 to 8.

Use of an antibody-drug conjugate for the manufacture of a medicament for treating a metastatic brain tumor, represented by the following formula:

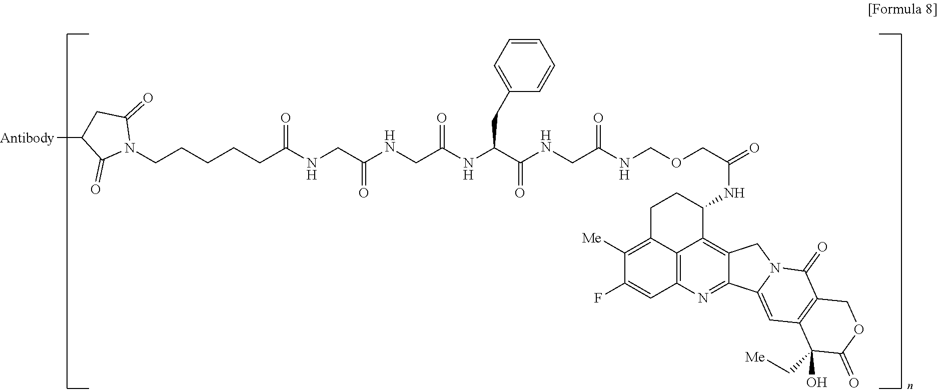

wherein the drug-linker is conjugated to the antibody via a thioether bond, and n is the average number of units of the drug-linker conjugated per antibody molecule.

The use according to [218], wherein the primary cancer for the metastatic brain tumor is at least one selected from the group consisting of breast cancer, lung cancer, melanoma, renal cell cancer, kidney cancer, colorectal cancer, gastric cancer, head and neck cancer, hepatocellular cancer, liver cancer, ovarian cancer, prostate cancer, bladder cancer, pancreatic cancer, endometrial cancer, thyroid cancer, malignant lymphoma and sarcoma.

The use according to [218], wherein the primary cancer for the metastatic brain tumor is at least one selected from the group consisting of breast cancer, lung cancer and melanoma.

The use according to [218], wherein the primary cancer for the metastatic brain tumor is breast cancer.

The use according to [218], wherein the primary cancer for the metastatic brain tumor is lung cancer.

The use according to [218], wherein the primary cancer for the metastatic brain tumor is melanoma.

The use according to any one of [218] to [223], wherein the antibody in the antibody-drug conjugate is an anti-HER2 antibody, an anti-HER3 antibody, an anti-TROP2 antibody, an anti-B7-H3 antibody, an anti-GPR20 antibody or an anti-CDH6 antibody.

The use according to [224], wherein the antibody in the antibody-drug conjugate is an anti-HER2 antibody.

The use according to [225], wherein the anti-HER2 antibody is an antibody comprising a heavy chain consisting of an amino acid sequence consisting of amino acid residues 1 to 449 of SEQ ID NO: 1 and a light chain consisting of an amino acid sequence consisting of amino acid residues 1 to 214 of SEQ ID NO: 2.

The use according to [225], wherein the anti-HER2 antibody is an antibody comprising a heavy chain consisting of an amino acid sequence represented by SEQ ID NO: 1 and a light chain consisting of an amino acid sequence represented by SEQ ID NO: 2.

The use according to any one of [225] to [227], wherein the average number of units of the drug-linker conjugated per antibody molecule in the antibody-drug conjugate is in the range of from 7 to 8.

The use according to [224], wherein the antibody in the antibody-drug conjugate is an anti-HER3 antibody.

The use according to [229], wherein the anti-HER3 antibody is an antibody comprising a heavy chain consisting of an amino acid sequence represented by SEQ ID NO: 3 and a light chain consisting of an amino acid sequence represented by SEQ ID NO: 4.

The use according to [230], wherein the anti-HER3 antibody lacks a lysine residue at the carboxyl terminus of the heavy chain.

The use according to any one of [229] to [231], wherein the average number of units of the drug-linker conjugated per antibody molecule in the antibody-drug conjugate is in the range of from 7 to 8.

The use according to [224], wherein the antibody in the antibody-drug conjugate is an anti-TROP2 antibody.

The use according to [233], wherein the anti-TROP2 antibody is an antibody comprising a heavy chain consisting of an amino acid sequence consisting of amino acid residues 20 to 470 of SEQ ID NO: 5 and a light chain consisting of an amino acid sequence consisting of amino acid residues 21 to 234 of SEQ ID NO: 6.

The use according to [234], wherein the anti-TROP2 antibody lacks a lysine residue at the carboxyl terminus of the heavy chain.

The use according to any one of [233] to [235], wherein the average number of units of the drug-linker conjugated per antibody molecule in the antibody-drug conjugate is in the range of from 3.5 to 4.5.

The use according to [224], wherein the antibody in the antibody-drug conjugate is an anti-B7-H3 antibody.

The use according to [237], wherein the anti-B7-H3 antibody is an antibody comprising a heavy chain consisting of an amino acid sequence consisting of amino acid residues 20 to 471 of SEQ ID NO: 7 and a light chain consisting of an amino acid sequence consisting of amino acid residues 21 to 233 of SEQ ID NO: 8.

The use according to [238], wherein the anti-B7-H3 antibody lacks a lysine residue at the carboxyl terminus of the heavy chain.

The use according to any one of [237] to [239], wherein the average number of units of the drug-linker conjugated per antibody molecule in the antibody-drug conjugate is in the range of from 3.5 to 4.5.

The use according to [224], wherein the antibody in the antibody-drug conjugate is an anti-GPR20 antibody.

The use according to [241], wherein the anti-GPR20 antibody is an antibody comprising a heavy chain consisting of an amino acid sequence consisting of amino acid residues 20 to 472 of SEQ ID NO: 9 and a light chain consisting of an amino acid sequence consisting of amino acid residues 21 to 234 of SEQ ID NO: 10.

The use according to [242], wherein the anti-GPR20 antibody lacks a lysine residue at the carboxyl terminus of the heavy chain.

The use according to any one of [241] to [243], wherein the average number of units of the drug-linker conjugated per antibody molecule in the antibody-drug conjugate is in the range of from 7 to 8.

The use according to [244], wherein the antibody in the antibody-drug conjugate is an anti-CDH6 antibody.

The use according to [245], wherein the anti-CDH6 antibody is an antibody comprising a heavy chain consisting of an amino acid sequence consisting of amino acid residues 20 to 471 of SEQ ID NO: 11 and a light chain consisting of an amino acid sequence consisting of amino acid residues 21 to 233 of SEQ ID NO: 12.

The use according to [246], wherein the anti-CDH6 antibody lacks a lysine residue at the carboxyl terminus of the heavy chain.

The use according to any one of [245] to [247], wherein the average number of units of the drug-linker conjugated per antibody molecule in the antibody-drug conjugate is in the range of from 7 to 8.

A therapeutic agent for metastatic cancer comprising, as an active component, an antibody-drug conjugate in which a drug-linker represented by the following formula:

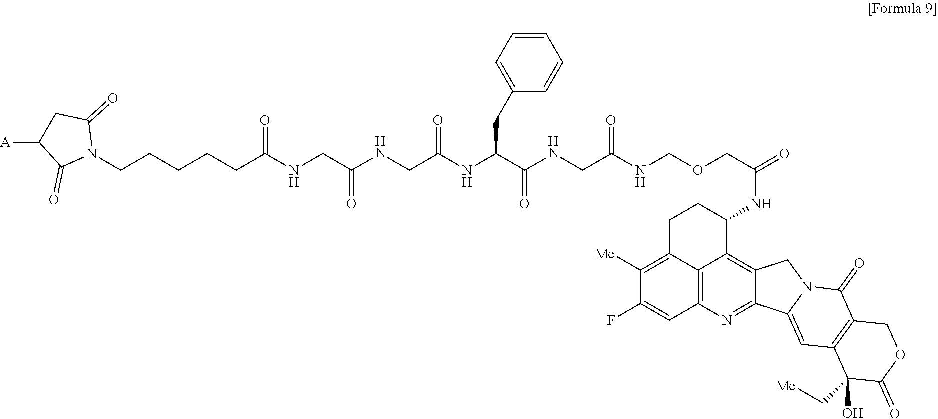

wherein A represents a connecting position to an antibody;
is conjugated to the antibody via a thioether bond.

The therapeutic agent according to [249], wherein the metastatic cancer is at least one selected from the group consisting of a metastatic brain tumor, a metastatic bone tumor, a metastatic lung tumor and metastatic liver cancer.

The therapeutic agent according to [250], wherein the metastatic cancer is a metastatic bone tumor.

A therapeutic agent for metastatic cancer comprising, as an active component, an antibody-drug conjugate represented by the following formula:

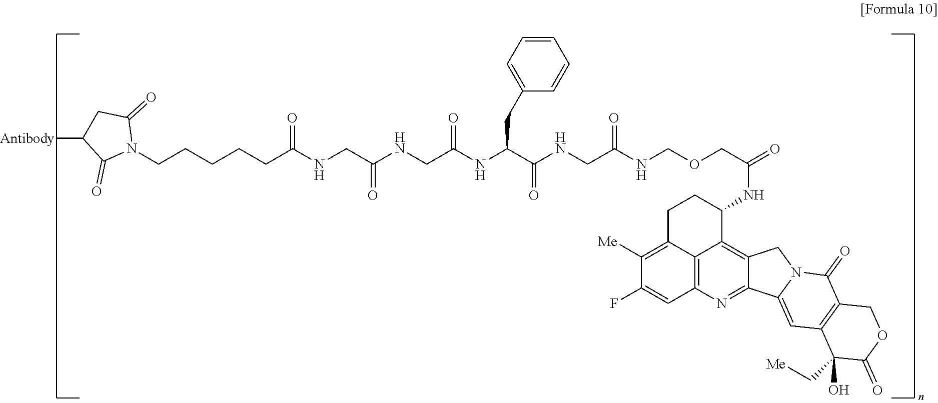

wherein the drug-linker is conjugated to the antibody via a thioether bond, and n is the average number of units of the drug-linker conjugated per antibody molecule.

The therapeutic agent according to [252], wherein the metastatic cancer is at least one selected from the group consisting of a metastatic brain tumor, a metastatic bone tumor, a metastatic lung tumor and metastatic liver cancer.

The therapeutic agent according to [253], wherein the metastatic cancer is a metastatic bone tumor.

A method of treatment for metastatic cancer, comprising administering an antibody-drug conjugate in which a drug-linker represented by the following formula:

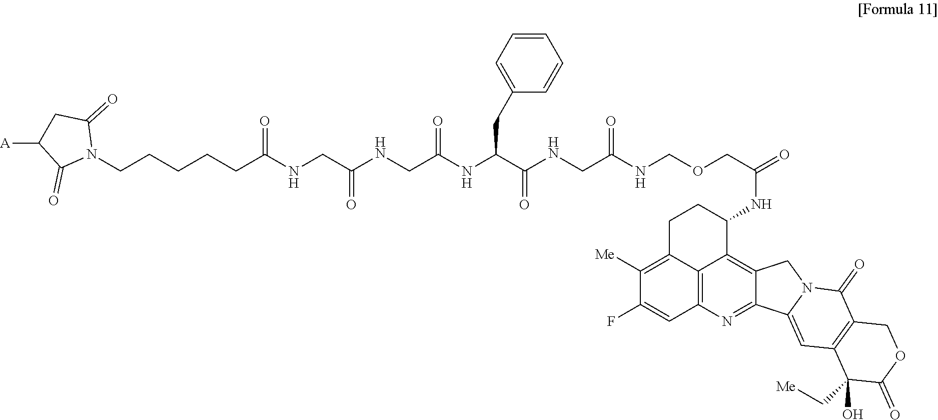

wherein A represents a connecting position to an antibody, is conjugated to the antibody via a thioether bond; to a subject in need of the treatment for metastatic cancer.

The method of treatment according to [255], wherein the metastatic cancer is at least one selected from the group consisting of a metastatic brain tumor, a metastatic bone tumor, a metastatic lung tumor and metastatic liver cancer.

The method of treatment according to [256], wherein the metastatic cancer is a metastatic bone tumor.

A method of treatment for metastatic cancer, comprising administering an antibody-drug conjugate represented by the following formula:

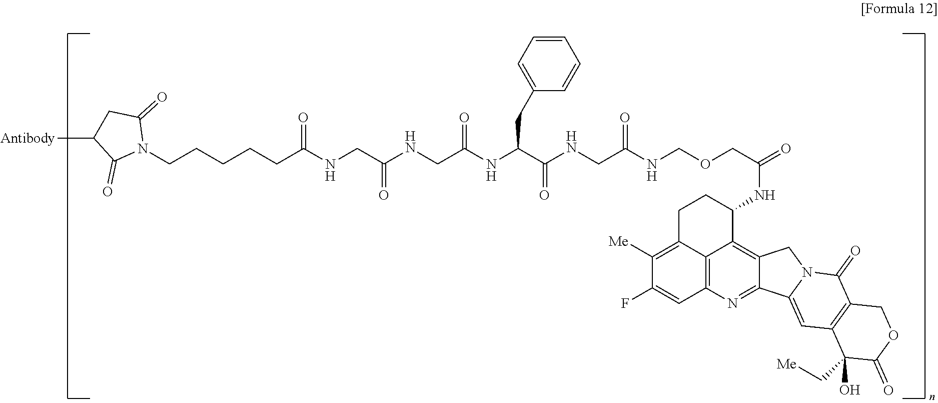

wherein the drug-linker is conjugated to the antibody via a thioether bond, and n is the average number of units of the drug-linker conjugated per antibody molecule;
to a subject in need of the treatment for metastatic cancer.

The method of treatment according to [258], wherein the metastatic cancer is at least one selected from the group consisting of a metastatic brain tumor, a metastatic bone tumor, a metastatic lung tumor and metastatic liver cancer.

The method of treatment according to [259], wherein the metastatic cancer is a metastatic bone tumor.

An antibody-drug conjugate for use in treating metastatic cancer, in which a drug-linker represented by the following formula:

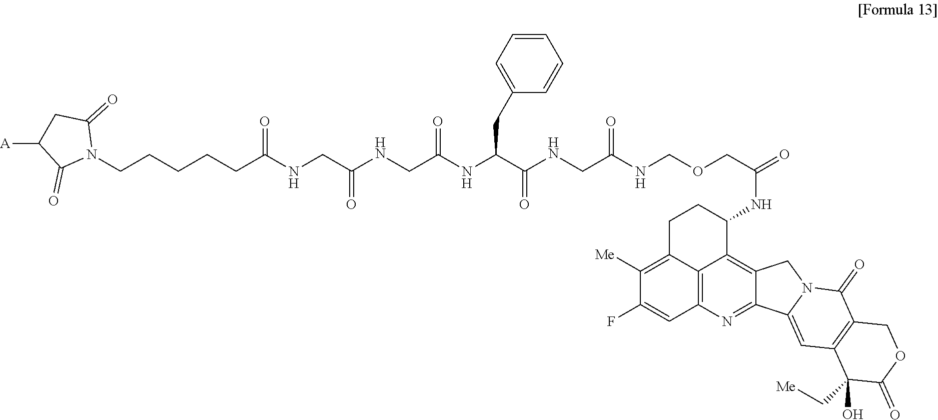

wherein A represents a connecting position to an antibody;
is conjugated to the antibody via a thioether bond.

The antibody-drug conjugate according to [261], wherein the metastatic cancer is at least one selected from the group consisting of a metastatic brain tumor, a metastatic bone tumor, a metastatic lung tumor and metastatic liver cancer.

The antibody-drug conjugate according to [262], wherein the metastatic cancer is a metastatic bone tumor.

An antibody-drug conjugate for use in treating metastatic cancer, represented by the following formula:

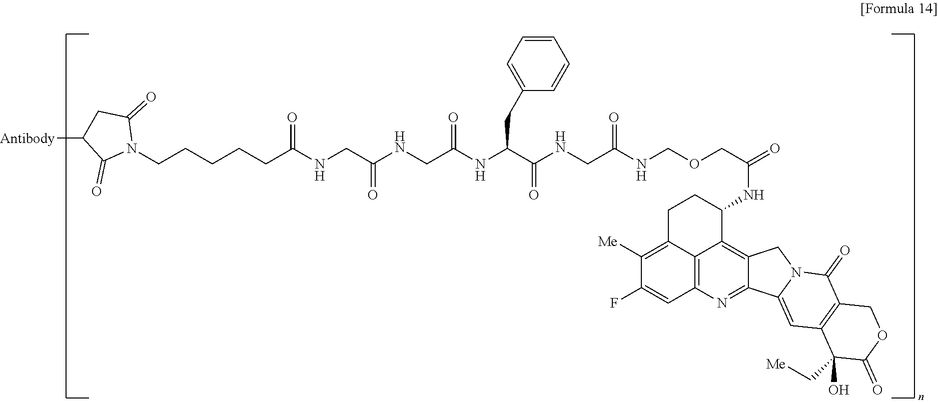

wherein the drug-linker is conjugated to the antibody via a thioether bond, and n is the average number of units of the drug-linker conjugated per antibody molecule.

The antibody-drug conjugate according to [264], wherein the metastatic cancer is at least one selected from the group consisting of a metastatic brain tumor, a metastatic bone tumor, a metastatic lung tumor and metastatic liver cancer.

The antibody-drug conjugate according to [265], wherein the metastatic cancer is a metastatic bone tumor.

Use of an antibody-drug conjugate for the manufacture of a medicament for treating metastatic cancer, in which a drug-linker represented by the following formula:

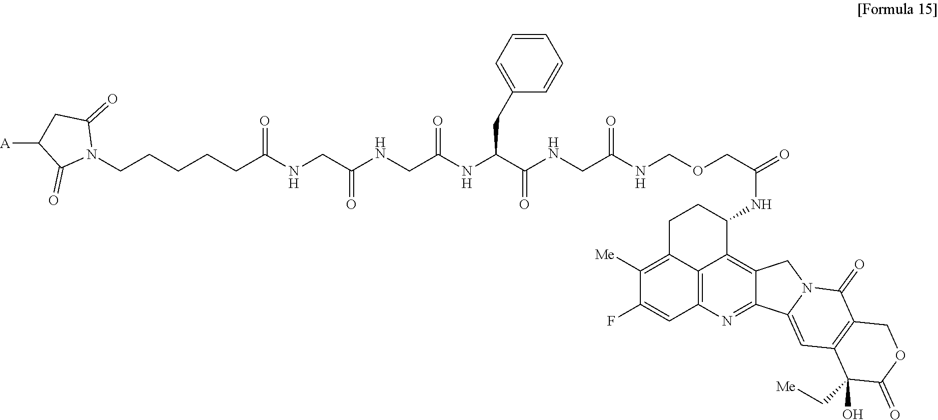

wherein A represents a connecting position to an antibody;
is conjugated to the antibody via a thioether bond.

The use according to [267], wherein the metastatic cancer is at least one selected from the group consisting of a metastatic brain tumor, a metastatic bone tumor, a metastatic lung tumor and metastatic liver cancer.

The use according to [268], wherein the metastatic cancer is a metastatic bone tumor.

Use of an antibody-drug conjugate for the manufacture of a medicament for treating metastatic cancer, represented by the following formula:

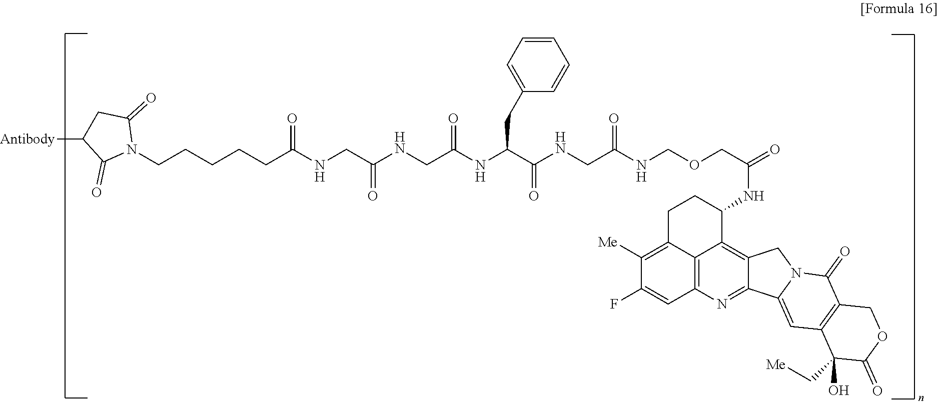

wherein the drug-linker is conjugated to the antibody via a thioether bond, and n is the average number of units of the drug-linker conjugated per antibody molecule.

The use according to [270], wherein the metastatic cancer is at least one selected from the group consisting of a metastatic brain tumor, a metastatic bone tumor, a metastatic lung tumor and metastatic liver cancer.

The use according to [271], wherein the metastatic cancer is a metastatic bone tumor.

### Advantageous Effects of Invention

The present invention provides a therapeutic agent for a metastatic brain tumor comprising a specific antibody-drug conjugate, and/or a method of treatment for a metastatic brain tumor comprising administering a specific antibody-drug conjugate to a subject.

### BRIEF DESCRIPTION OF DRAWINGS

FIG. 1 is a diagram showing the amino acid sequence of a heavy chain of an anti-HER2 antibody (SEQ ID NO: 1).

FIG. 2 is a diagram showing the amino acid sequence of a light chain of an anti-HER2 antibody (SEQ ID NO: 2).

FIG. 3 is a diagram showing the amino acid sequence of a heavy chain of an anti-HER3 antibody (SEQ ID NO: 3).

FIG. 4 is a diagram showing the amino acid sequence of a light chain of an anti-HER3 antibody (SEQ ID NO: 4).

FIG. 5 is a diagram showing the amino acid sequence of a heavy chain of an anti-TROP2 antibody (SEQ ID NO: 5).

FIG. 6 is a diagram showing the amino acid sequence of a light chain of an anti-TROP2 antibody (SEQ ID NO: 6).

FIG. 7 is a diagram showing the amino acid sequence of a heavy chain of an anti-B7-H3 antibody (SEQ ID NO: 7).

FIG. 8 is a diagram showing the amino acid sequence of a light chain of an anti-B7-H3 antibody (SEQ ID NO: 8).

FIG. 9 is a diagram showing the life-prolonging effects of an antibody-drug conjugate (1) on mice with intracranially transplanted KPL-4-Luc.

FIG. 10 is a diagram showing the antitumor effects of an antibody-drug conjugate (1) on mice with intracranially transplanted KPL-4-Luc. The amount of tumor is confirmed by the luminescence intensity of KPL-4-Luc.

FIG. 11 is diagrams showing brain pathological images by hematoxylin and eosin staining on mice (No.1 and No.2) of a solvent-administered group. The center images show overall pathological images, and the left and right images show enlarged pathological images. Tumor masses are found in the area surrounded by a dotted line or the area shown by arrows.

FIG. 12 is a diagram showing the amino acid sequence of a heavy chain of an anti-GPR20 antibody (SEQ ID NO: 9).

FIG. 13 is a diagram showing the amino acid sequence of a light chain of an anti-GPR20 antibody (SEQ ID NO: 10).

FIG. 14 is a diagram showing the amino acid sequence of a heavy chain of an anti-CDH6 antibody (SEQ ID NO: 11).

FIG. 15 is a diagram showing the amino acid sequence of a light chain of an anti-CDH6 antibody (SEQ ID NO: 12).

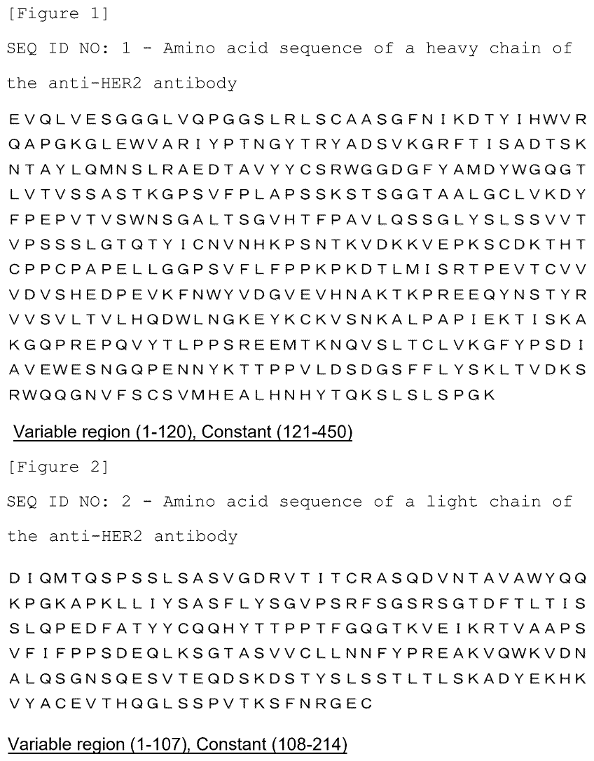

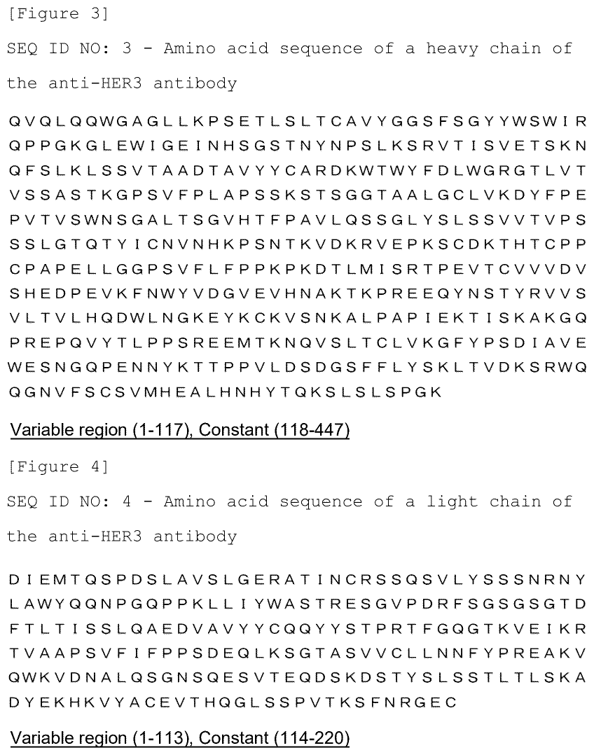

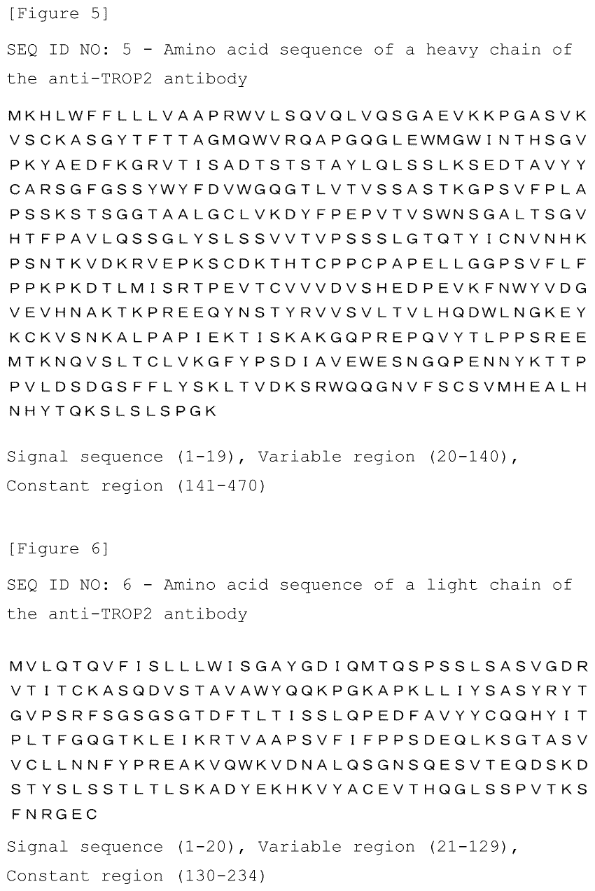

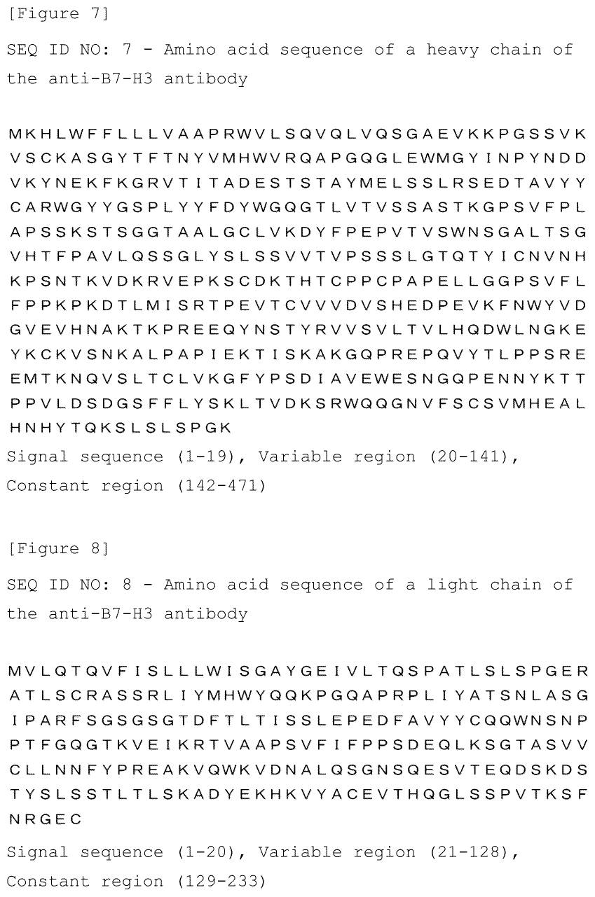

### DESCRIPTION OF EMBODIMENTS

Hereinafter, preferred modes for carrying out the present invention are described. The embodiments described below are given merely for illustrating one example of a typical embodiment of the present invention and are not intended to limit the scope of the present invention.

### 1. Metastatic Brain Tumor

In the present invention, the term “metastatic brain tumor” means a tumor developed by metastasis of a primary cancer (a cancer developed in biological tissues other than the brain) to the brain. The metastatic brain tumor in the present invention includes not only tumors metastasized into the brain parenchyma, but also tumors invading the pia mater and the arachnoid membrane (meningeal carcinomatosis). The metastasis site may be one or several sites. Further, the metastatic brain tumor is mostly found after symptoms of a primary cancer appear, but may also be found before symptoms of a primary cancer appear.

As the symptoms of the metastatic brain tumor, for example, headache, vomiting, visual impairment, consciousness disorder, convulsive seizure, paralysis and language disorder can be exemplified. These symptoms are inferred to be caused by direct damage of brain tissues by a tumor and an increased intracranial pressure.

Examination of the metastatic brain tumor can be carried out by, for example, CT (Computed Tomography), PET (Positron Emission Tomography), MRI (Magnetic Resonance Imaging), or the like.

For the primary cancer for metastatic brain tumors, lung cancer, breast cancer, melanoma, renal cell cancer, kidney cancer, colorectal cancer, gastric cancer, head and neck cancer, hepatocellular cancer, liver cancer, ovarian cancer, prostate cancer, bladder cancer, pancreatic cancer, endometrial cancer, thyroid cancer, malignant lymphoma and sarcoma can be exemplified. Among these, lung cancer, breast cancer and melanoma are particularly found as primary cancers for metastatic brain tumors at high percentages.

2. Antibody-drug conjugate

The antibody-drug conjugate used in the present invention is an antibody-drug conjugate in which a drug-linker represented by the following formula:

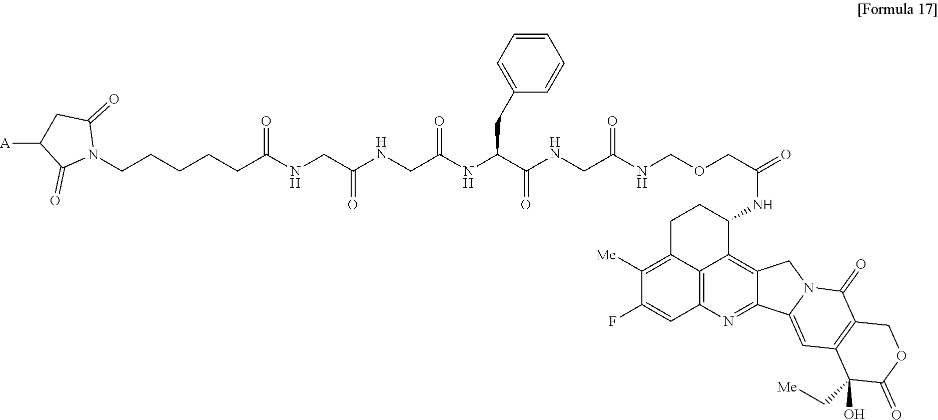

wherein A represents a connecting position to an antibody;
   is conjugated to the antibody via a thioether bond.

In the present invention, the partial structure consisting of a linker and a drug in the antibody-drug conjugate is referred to as a “drug-linker”. The drug-linker is connected to a thiol group (in other words, the sulfur atom of a cysteine residue) formed at an interchain disulfide bond site (two sites between heavy chains, and two sites between a heavy chain and a light chain) in the antibody.

The drug-linker of the present invention includes exatecan (IUPAC name: (1S, 9S)-1-amino-9-ethyl-5-fluoro-1, 2, 3, 9, 12, 15-hexahydro-9-hydroxy-4-methyl-10H, 13H-benzo[de]pyrano[3′, 4′: 6,7] indolizino[1,2-b]quinolin-10,13-dione, (also expressed as chemical name: (1S, 9S)-1-amino-9-ethyl-5-fluoro-2, 3-dihydro-9-hydroxy-4-methyl-1H, 12H-benzo[de] pyrano[3′, 4′: 6,7] indolizino[1,2-b]quinolin-10,13 (9H, 15H)-dione)), which is a topoisomerase I inhibitor, as a component. Exatecan is a camptothecin derivative having an antitumor effect, represented by the following formula:

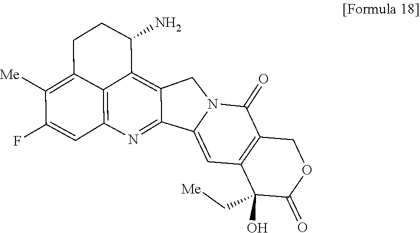 [SMILES: CC[C@@]1(O)C(=O)OCc2c1cc1n(c2=O)Cc2c-1nc1cc(F)c(C)c3c1c2[C@@H](N)CC3]

The antibody-drug conjugate used in the present invention can also be represented by the following formula:

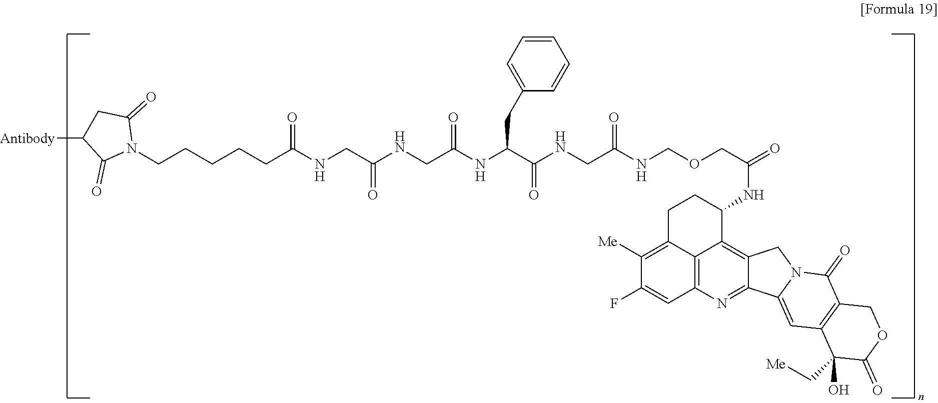

wherein, the drug-linker is conjugated to an antibody via a thioether bond. The meaning of n is the same as that of what is called the average number of conjugated drug molecules (DAR; Drug-to-Antibody Ratio), and indicates the average number of units of the drug-linker conjugated per antibody molecule.

After migrating into cancer cells, the antibody-drug conjugate used in the present invention releases the compound represented by the following formula:

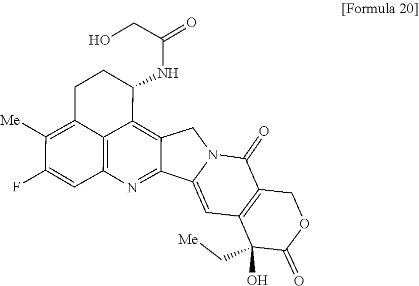 [SMILES: CC[C@@]1(O)C(=O)OCc2c1cc1n(c2=O)Cc2c-1nc1cc(F)c(C)c3c1c2[C@@H](NC(=O)CO)CC3]

and thereby exerts an antitumor effect.

The aforementioned compound is inferred to be the original source of the antitumor activity of the antibody-drug conjugate used in the present invention, and has been confirmed to have a topoisomerase I inhibitory effect (Ogitani Y. et al., Clinical Cancer Research, 2016 October 15; 22 (20): 5097-5108, Epub 2016 Mar. 29).

The antibody-drug conjugate used in the present invention is also known to have a bystander effect (Ogitani Y. et al., Cancer Science (2016) 107, 1039-1046).

The bystander effect is exerted through a process such that the antibody-drug conjugate used in the present invention is internalized in cancer cells expressing the target and the aforementioned compound is released and then exerts an antitumor effect also on cancer cells which are present therearound and not expressing the target.

### 3. Antibody in Antibody-Drug Conjugate

The antibody in the antibody-drug conjugate used in the present invention may be derived from any species, and is preferably an antibody derived from a human, a rat, a mouse, or a rabbit. In cases when the antibody is derived from species other than human species, it is preferably chimerized or humanized using a well-known technique. The antibody of the present invention may be a polyclonal antibody or a monoclonal antibody and is preferably a monoclonal antibody.

The antibody in the antibody-drug conjugate used in the present invention is an antibody preferably having the characteristic of being able to target cancer cells, and is preferably an antibody possessing the property of being able to recognize a cancer cell, the property of being able to bind to a cancer cell, the property of being incorporated and internalized in a cancer cell, and/or cytocidal activity against cancer cells.

The binding activity of the antibody against cancer cells can be confirmed using flow cytometry. The internalization of the antibody into cancer cells can be confirmed using (1) an assay of visualizing an antibody incorporated in cells under a fluorescence microscope using a secondary antibody (fluorescently labeled) binding to the therapeutic antibody (Cell Death and Differentiation (2008) 15, 751-761), (2) an assay of measuring a fluorescence intensity incorporated in cells using a secondary antibody (fluorescently labeled) binding to the therapeutic antibody (Molecular Biology of the Cell, Vol. 15, 5268-5282 Dec. 2004), or (3) a Mab-ZAP assay using an immunotoxin binding to the therapeutic antibody wherein the toxin is released upon incorporation into cells to inhibit cell growth (Bio Techniques 28:162-165, January 2000). As the immunotoxin, a recombinant complex protein of a diphtheria toxin catalytic domain and protein G may be used.

The antitumor activity of the antibody can be confirmed in vitro by determining inhibitory activity against cell growth. For example, a cancer cell line overexpressing a target protein for the antibody is cultured, and the antibody is added at varying concentrations into the culture system to be able to determine inhibitory activity against focus formation, colony formation, and spheroid growth. The antitumor activity can be confirmed in vivo, for example, by administering the antibody to a nude mouse with a transplanted cancer cell line highly expressing the target protein, and determining changes in the cancer cells.

Since the compound conjugated in the antibody-drug conjugate exerts an antitumor effect, it is preferred but not essential that the antibody itself should have an antitumor effect. For the purpose of specifically and selectively exerting the cytotoxic activity of the antitumor compound against cancer cells, it is important and also preferred that the antibody should have the property of being internalized to migrate into cancer cells.

The antibody in the antibody-drug conjugate used in the present invention can be obtained by a procedure known in the art. For example, the antibody of the present invention can be obtained using a method usually carried out in the art, which involves immunizing animals with an antigenic polypeptide and collecting and purifying antibodies produced in vivo. The origin of the antigen is not limited to humans, and the animals may be immunized with an antigen derived from a non-human animal such as a mouse, a rat and the like. In this case, the cross-reactivity of antibodies binding to the obtained heterologous antigen with human antigens can be tested to screen for an antibody applicable to a human disease.

Alternatively, antibody-producing cells which produce antibodies against the antigen can be fused with myeloma cells according to a method known in the art (e.g., Kohler and Milstein, Nature (1975) 256, p. 495-497; Kennet, R. ed., Monoclonal Antibodies, p. 365-367, Plenum Press, N.Y. (1980)) to establish hybridomas, from which monoclonal antibodies can in turn be obtained.

The antigen can be obtained by genetically engineering host cells to produce a gene encoding the antigenic protein. Specifically, vectors that permit expression of the antigen gene are prepared and transferred to host cells so that the gene is expressed. The antigen thus expressed can be purified. The antibody can also be obtained by a method of immunizing animals with the above-described genetically engineered antigen-expressing cells or a cell line expressing the antigen.

The antibody in the antibody-drug conjugate used in the present invention is preferably a recombinant antibody obtained by artificial modification for the purpose of decreasing heterologous antigenicity to humans such as a chimeric antibody or a humanized antibody, or is preferably an antibody having only the gene sequence of an antibody derived from a human, that is, a human antibody. These antibodies can be produced using a known method.

As the chimeric antibody, an antibody in which antibody variable and constant regions are derived from different species, for example, a chimeric antibody in which a mouse-or rat-derived antibody variable region is connected to a human-derived antibody constant region can be exemplified (Proc. Natl. Acad. Sci. USA, 81, 6851-6855, (1984)).

As the humanized antibody, an antibody obtained by integrating only the complementarity determining region (CDR) of a heterologous antibody into a human-derived antibody (Nature (1986) 321, pp. 522-525), an antibody obtained by grafting a part of the amino acid residues of the framework of a heterologous antibody as well as the CDR sequence of the heterologous antibody to a human antibody by a CDR-grafting method (WO 90/07861), and an antibody humanized using a gene conversion mutagenesis strategy (U.S. Pat. No. 5,821,337) can be exemplified.

As the human antibody, an antibody generated by using a human antibody-producing mouse having a human chromosome fragment including genes of a heavy chain and a light chain of a human antibody (see Tomizuka, K. et al., Nature Genetics (1997) 16, p.133-143; Kuroiwa, Y. et. al., Nucl. Acids Res. (1998) 26, p. 3447-3448; Yoshida, H. et. al., Animal Cell Technology: Basic and Applied Aspects vol. 10, p. 69-73 (Kitagawa, Y., Matsuda, T. and Iijima, S. eds.), Kluwer Academic Publishers, 1999; Tomizuka, K. et. al., Proc. Natl. Acad. Sci. USA (2000) 97, p. 722-727, etc.) can be exemplified. As an alternative, an antibody obtained by phage display, the antibody being selected from a human antibody library (see Wormstone, I. M. et. al, Investigative Ophthalmology & Visual Science. (2002) 43 (7), p.2301-2308; Carmen, S. et. al., Briefings in Functional Genomics and Proteomics (2002), 1 (2), p.189-203; Siriwardena, D. et. al., Ophthalmology (2002) 109 (3), p. 427-431, etc.) can be exemplified.

In the antibody in the antibody-drug conjugate used in present invention, modified variants of the antibody are also included. The modified variant refers to a variant obtained by subjecting the antibody according to the present invention to chemical or biological modification. Examples of the chemically modified variant include variants including a linkage of a chemical moiety to an amino acid skeleton, variants including a linkage of a chemical moiety to an N-linked or O-linked carbohydrate chain, etc. Examples of the biologically modified variant include variants obtained by post-translational modification (such as N-linked or O-linked glycosylation, N-or C-terminal processing, deamidation, isomerization of aspartic acid, or oxidation of methionine), and variants in which a methionine residue has been added to the N terminus by being expressed in a prokaryotic host cell. Further, an antibody labeled so as to enable the detection or isolation of the antibody or an antigen according to the present invention, for example, an enzyme-labeled antibody, a fluorescence-labeled antibody, and an affinity-labeled antibody are also included in the meaning of the modified variant. Such a modified variant of the antibody according to the present invention is useful for improving the stability and blood retention of the antibody, reducing the antigenicity thereof, detecting or isolating an antibody or an antigen, and so on.

Further, by regulating the modification of a glycan which is linked to the antibody according to the present invention (glycosylation, defucosylation, etc.), it is possible to enhance antibody-dependent cellular cytotoxic activity. As the technique for regulating the modification of a glycan of antibodies, WO 99/54342, WO 00/61739, WO 02/31140, WO 2007/133855, WO 2013/120066, etc. are known. However, the technique is not limited thereto. In the antibody according to the present invention, antibodies in which the modification of a glycan is regulated are also included.

It is known that a lysine residue at the carboxyl terminus of the heavy chain of an antibody produced in a cultured mammalian cell is deleted (Journal of Chromatography A, 705:129-134 (1995)), and it is also known that two amino acid residues (glycine and lysine) at the carboxyl terminus of the heavy chain of an antibody produced in a cultured mammalian cell are deleted and a proline residue newly located at the carboxyl terminus is amidated (Analytical Biochemistry, 360:75-83 (2007)). However, such deletion and modification of the heavy chain sequence do not affect the antigen-binding affinity and the effector function (complement activation, antibody-dependent cellular cytotoxicity, etc.) of the antibody. Therefore, in the antibody according to the present invention, antibodies subjected to such modification and functional fragments of the antibody are also included, and deletion variants in which one or two amino acids have been deleted at the carboxyl terminus of the heavy chain, variants obtained by amidation of the deletion variants (for example, a heavy chain in which the carboxyl terminal proline residue has been amidated), and the like are also included. The type of deletion variant having a deletion at the carboxyl terminus of the heavy chain of the antibody according to the present invention is not limited to the above variants as long as the antigen-binding affinity and the effector function are conserved. The two heavy chains constituting the antibody according to the present invention may be of one type selected from the group consisting of a full-length heavy chain and the above-described deletion variant, or may be of two types in combination selected therefrom. The ratio of the amount of each deletion variant can be affected by the type of cultured mammalian cells which produce the antibody according to the present invention and the culture conditions; however, an antibody in which one amino acid residue at the carboxyl terminus has been deleted in both of the two heavy chains in the antibody according to the present invention can be preferably exemplified.

As isotypes of the antibody according to the present invention, for example, IgG (IgG1, IgG2, IgG3, IgG4) can be exemplified, and IgG1 or IgG2 can be exemplified preferably.

Examples of antibodies in the antibody-drug conjugate used in the present invention can include, but are not particularly limited to, an anti-HER2 antibody, an anti-HER3 antibody, an anti-TROP2 antibody, an anti-B7-H3 antibody, an anti-CD3 antibody, an anti-CD30 antibody, an anti-CD33 antibody, an anti-CD37 antibody, an anti-CD56 antibody, an anti-CD98 antibody, an anti-DR5 antibody, an anti-EGFR antibody, an anti-EPHA2 antibody, an anti-FGFR2 antibody, an anti-FGFR4 antibody, an anti-FOLR1 antibody, an anti-VEGF antibody, an anti-CD20 antibody, an anti-CD22 antibody, an anti-CD70 antibody, an anti-PSMA antibody, an anti-CEA antibody, an anti-Mesothelin antibody, an anti-A33 antibody, an anti-CanAg antibody, an anti-Cripto antibody, an anti-G250 antibody, an anti-MUC1 antibody, an anti-GPNMB antibody, an anti-Integrin antibody, an anti-Tenascin-C antibody, an anti-SLC44A4 antibody, an anti-GPR20 antibody, and an anti-CDH6 antibody. Further, an anti-HER2 antibody, an anti-HER3 antibody, an anti-TROP2 antibody, an anti-B7-H3 antibody, an anti-GPR20 antibody, and an anti-CDH6 antibody can be preferably exemplified, and an anti-HER2 antibody can be more preferably exemplified.

In the present invention, the term “anti-HER2 antibody” refers to an antibody which binds specifically to HER2 (Human Epidermal Growth Factor Receptor Type 2; ErbB-2), and preferably has an activity of internalization in HER2-expressing cells by binding to HER2.

Examples of the anti-HER2 antibody include trastuzumab (U.S. Pat. No. 5,821,337) and pertuzumab (International Publication No. WO 01/00245). Preferably, trastuzumab can be exemplified.

In the present invention, the term “anti-HER3 antibody” refers to an antibody which binds specifically to HER3 (Human Epidermal Growth Factor Receptor Type 3; ErbB-3), and preferably has an activity of internalization in HER3-expressing cells by binding to HER3.

Examples of the anti-HER3 antibody include patritumab (U3-1287), U1-59 (International Publication No. WO 2007/077028), MM-121 (seribantumab), an anti-ERBB3 antibody described in International Publication No. WO 2008/100624, RG-7116 (lumretuzumab), and LJM-716 (elgemtumab). Preferably, patritumab and U1-59 can be exemplified.

In the present invention, the term “anti-TROP2 antibody” refers to an antibody which binds specifically to TROP2 (TACSTD2: Tumor-associated calcium signal transducer 2; EGP-1), and preferably has an activity of internalization in TROP2-expressing cells by binding to TROP2.

Examples of the anti-TROP2 antibody include hTINA1-H1L1 (International Publication No. WO 2015/098099).

In the present invention, the term “anti-B7-H3 antibody” refers to an antibody which binds specifically to B7-H3 (B cell antigen #7 homolog 3; PD-L3; CD276), and preferably has an activity of internalization in B7-H3-expressing cells by binding to B7-H3.

Examples of the anti-B7-H3 antibody include M30-H1-L4 (International Publication No. WO 2014/057687).

In the present invention, the term “anti-GPR20 antibody” refers to an antibody which binds specifically to GPR20 (G Protein-coupled receptor 20), and preferably has an activity of internalization in GPR20-expressing cells by binding to GPR20.

Examples of the anti-GPR20 antibody include h046-H4e/L7 (International Publication No. WO 2018/135501).

In the present invention, the term “anti-CDH6 antibody” refers to an antibody which binds specifically to CDH6 (Cadherin-6), and preferably has an activity of internalization in CDH6-expressing cells by binding to CDH6.

Examples of the anti-CDH6 antibody include H01L02 (International Publication No. WO 2018/212136).

### 4. Production of Antibody-Drug Conjugate

A drug-linker intermediate for use in the production of the antibody-drug conjugate used in the present invention is represented by the following formula.

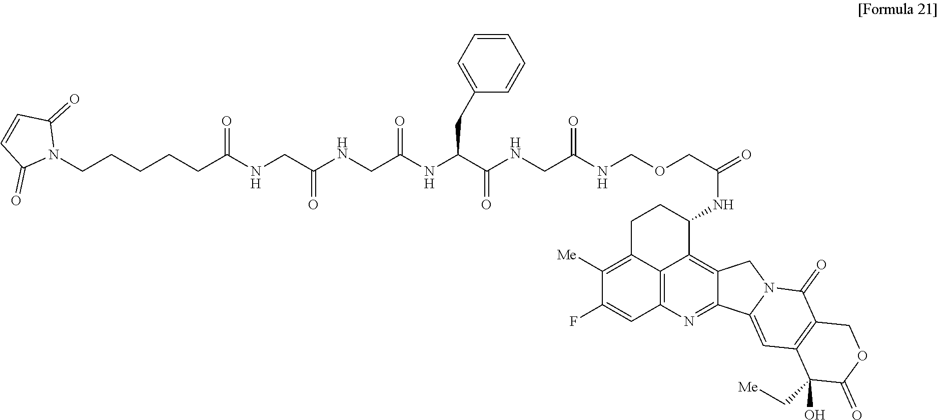 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCCCCN1C(=O)C=CC1=O)CC3]

The drug-linker intermediate can be expressed as the chemical name N-[6-(2,5-dioxo-2,5-dihydro-1H-pyrrol-1-yl) hexanoyl]glycylglycyl-L-phenylalanyl-N-[(2-{[(1, 9S)-9-ethyl-5-fluoro-9-hydroxy-4-methyl-10,13-dioxo-2, 3, 9, 10, 13, 15-hexahydro-1H, 12H-benzo[de] pyrano[3′, 4′: 6,7] indolizino[1, 2-b] quinolin-1-yl]amino}-2-oxoethoxy)methyl] glycinamide, and can be produced with reference to descriptions in International Publication No. WO 2014/057687, International Publication No. WO 2015/098099, International Publication No. WO 2015/115091, International Publication No. WO 2015/155998, International Publication No. WO 2019/044947, and so on.

The antibody-drug conjugate used in the present invention can be produced by reacting the above-described drug-linker intermediate and an antibody having a thiol group (alternatively referred to as a sulfhydryl group).

The antibody having a sulfhydryl group can be obtained by a method well known in the art (Hermanson, G. T, Bioconjugate Techniques, pp. 56-136, pp. 456-493, Academic Press (1996)). For example, by using 0.3 to 3 molar equivalents of a reducing agent such as tris(2-carboxyethyl) phosphine hydrochloride (TCEP) per interchain disulfide within the antibody and reacting with the antibody in a buffer solution containing a chelating agent such as ethylenediamine tetraacetic acid (EDTA), an antibody having a sulfhydryl group with partially or completely reduced interchain disulfides within the antibody can be obtained.

Further, by using 2 to 20 molar equivalents of the drug-linker intermediate per the antibody having a sulfhydryl group, an antibody-drug conjugate in which 2 to 8 drug molecules are conjugated per antibody molecule can be produced.

The average number of conjugated drug molecules per antibody molecule of the antibody-drug conjugate produced can be determined, for example, by a method of calculation based on measurement of UV absorbance for the anti-HER2 antibody-drug conjugate and the conjugation precursor thereof at two wavelengths of 280 nm and 370 nm (UV method), or a method of calculation based on quantification through HPLC measurement for fragments obtained by treating the antibody-drug conjugate with a reducing agent (HPLC method).

Conjugation between the antibody and the drug-linker intermediate and calculation of the average number of conjugated drug molecules per antibody molecule of the antibody-drug conjugate can be performed with reference to descriptions in International Publication No. WO 2014/057687, International Publication No. WO 2015/098099, International Publication No. WO 2015/115091, International Publication No. WO 2015/155998, International Publication No. WO 2018/135501, International Publication No. WO 2018/212136, and so on.

In the present invention, the term “anti-HER2 antibody-drug conjugate” refers to an antibody-drug conjugate such that the antibody in the antibody-drug conjugate according to the present invention is an anti-HER2 antibody.

The anti-HER2 antibody is preferably an antibody comprising a heavy chain consisting of an amino acid sequence consisting of amino acid residues 1 to 449 of SEQ ID NO: 1 and a light chain consisting of an amino acid sequence consisting of amino acid residues 1 to 214 of SEQ ID NO: 2, or an antibody comprising a heavy chain consisting of an amino acid sequence represented by SEQ ID NO: 1 and a light chain consisting of an amino acid sequence represented by SEQ ID NO: 2.

The average number of units of the drug-linker conjugated per antibody molecule in the anti-HER2 antibody-drug conjugate is preferably 2 to 8, more preferably 3 to 8, even more preferably 7 to 8, even more preferably 7.5 to 8, and even more preferably about 8.

The anti-HER2 antibody-drug conjugate can be produced with reference to descriptions in International Publication No. WO 2015/115091 and so on.

In the present invention, the term “anti-HER3 antibody-drug conjugate” refers to an antibody-drug conjugate such that the antibody in the antibody-drug conjugate according to the present invention is an anti-HER3 antibody.

The anti-HER3 antibody is preferably an antibody comprising a heavy chain comprising CDRH1 consisting of an amino acid sequence consisting of amino acid residues 26 to 35 of SEQ ID NO: 3, CDRH2 consisting of an amino acid sequence consisting of amino acid residues 50 to 65 of SEQ ID NO: 3, and CDRH3 consisting of an amino acid sequence consisting of amino acid residues 98 to 106 of SEQ ID NO: 3 and a light chain comprising CDRL1 consisting of an amino acid sequence consisting of amino acid residues 24 to 39 of SEQ ID NO: 4, CDRL2 consisting of an amino acid sequence consisting of amino acid residues 56 to 62 of SEQ ID NO: 4, and CDRL3 consisting of an amino acid sequence consisting of amino acid residues 95 to 103 of SEQ ID NO: 4,
more preferably an antibody comprising a heavy chain comprising a heavy chain variable region consisting of an amino acid sequence consisting of amino acid residues 1 to 117 of SEQ ID NO: 3 and a light chain comprising a light chain variable region consisting of an amino acid sequence consisting of amino acid residues 1 to 113 of SEQ ID NO: 4, and
even more preferably an antibody comprising a heavy chain consisting of an amino acid sequence represented by SEQ ID NO: 3 and a light chain consisting of an amino acid sequence represented by SEQ ID NO: 4, or a variant of the antibody in which a lysine residue at the carboxyl terminus of the heavy chain is deleted.

The average number of units of the drug-linker conjugated per antibody molecule in the anti-HER3 antibody-drug conjugate is preferably 2 to 8, more preferably 3 to 8, even more preferably 7 to 8, even more preferably 7.5 to 8, and even more preferably about 8.

The anti-HER3 antibody-drug conjugate can be produced with reference to descriptions in International Publication No. WO 2015/155998 and so on.

In the present invention, the term “anti-TROP2 antibody-drug conjugate” refers to an antibody-drug conjugate such that the antibody in the antibody-drug conjugate according to the present invention is an anti-TROP2 antibody.

The anti-TROP2 antibody is preferably an antibody comprising a heavy chain comprising CDRH1 consisting of an amino acid sequence consisting of amino acid residues 50 to 54 of SEQ ID NO: 5, CDRH2 consisting of an amino acid sequence consisting of amino acid residues 69 to 85 of SEQ ID NO: 5, and CDRH3 consisting of an amino acid sequence consisting of amino acid residues 118 to 129 of SEQ ID NO: 5 and a light chain comprising CDRL1 consisting of an amino acid sequence consisting of amino acid residues 44 to 54 of SEQ ID NO: 6, CDRL2 consisting of an amino acid sequence consisting of amino acid residues 70 to 76 of SEQ ID NO: 6, and CDRL3 consisting of an amino acid sequence consisting of amino acid residues 109 to 117 of SEQ ID NO: 6,
more preferably an antibody comprising a heavy chain comprising a heavy chain variable region consisting of an amino acid sequence consisting of amino acid residues 20 to 140 of SEQ ID NO: 5 and a light chain comprising a light chain variable region consisting of an amino acid sequence consisting of amino acid residues 21 to 129 of SEQ ID NO: 6, and
even more preferably an antibody comprising a heavy chain consisting of an amino acid sequence consisting of amino acid residues 20 to 470 of SEQ ID NO: 5 and a light chain consisting of an amino acid sequence consisting of amino acid residues 21 to 234 of SEQ ID NO: 6, or a variant of the antibody in which a lysine residue at the carboxyl terminus of the heavy chain is deleted.

The average number of units of the drug-linker conjugated per antibody molecule in the anti-TROP2 antibody-drug conjugate is preferably 2 to 8, more preferably 3 to 5, even more preferably 3.5 to 4.5, and even more preferably about 4.

The anti-TROP2 antibody-drug conjugate can be produced with reference to descriptions in International Publication No. WO 2015/098099 and so on.

In the present invention, the term “anti-B7-H3 antibody-drug conjugate” refers to an antibody-drug conjugate such that the antibody in the antibody-drug conjugate according to the present invention is an anti-B7-H3 antibody.

The anti-B7-H3 antibody is preferably an antibody comprising a heavy chain comprising CDRH1 consisting of an amino acid sequence consisting of amino acid residues 50 to 54 of SEQ ID NO: 7, CDRH2 consisting of an amino acid sequence consisting of amino acid residues 69 to 85 of SEQ ID NO: 7, and CDRH3 consisting of an amino acid sequence consisting of amino acid residues 118 to 130 of SEQ ID NO: 7 and a light chain comprising CDRL1 consisting of an amino acid sequence consisting of amino acid residues 44 to 53 of SEQ ID NO: 8, CDRL2 consisting of an amino acid sequence consisting of amino acid residues 69 to 75 of SEQ ID NO: 8, and CDRL3 consisting of an amino acid sequence consisting of amino acid residues 108 to 116 of SEQ ID NO: 8, more preferably an antibody comprising a heavy chain comprising a heavy chain variable region consisting of an amino acid sequence consisting of amino acid residues 20 to 141 of SEQ ID NO: 7 and a light chain comprising a light chain variable region consisting of an amino acid sequence consisting of amino acid residues 21 to 128 of SEQ ID NO: 8, and even more preferably an antibody comprising a heavy chain consisting of an amino acid sequence consisting of amino acid residues 20 to 471 of SEQ ID NO: 7 and a light chain consisting of an amino acid sequence consisting of amino acid residues 21 to 233 of SEQ ID NO: 8, or a variant of the antibody in which a lysine residue at the carboxyl terminus of the heavy chain is deleted.

The average number of units of the drug-linker conjugated per antibody molecule in the anti-B7-H3 antibody-drug conjugate is preferably 2 to 8, more preferably 3 to 5, even more preferably 3.5 to 4.5, and even more preferably about 4.

The anti-B7-H3 antibody-drug conjugate used in the present invention can be produced with reference to descriptions in International Publication No. WO 2014/057687 and so on.

In the present invention, the term “anti-GPR20 antibody-drug conjugate” refers to an antibody-drug conjugate wherein the antibody in the antibody-drug conjugate according to the present invention is an anti-GPR20 antibody.

The anti-GPR20 antibody is preferably an antibody comprising a heavy chain comprising CDRH1 consisting of an amino acid sequence consisting of amino acid residues 45 to 54 of SEQ ID NO: 9, CDRH2 consisting of an amino acid sequence consisting of amino acid residues 69 to 78 of SEQ ID NO: 9, and CDRH3 consisting of an amino acid sequence consisting of amino acid residues 118 to 131 of SEQ ID NO: 9 and a light chain comprising CDRL1 consisting of an amino acid sequence consisting of amino acid residues 44 to 54 of SEQ ID NO: 10, CDRL2 consisting of an amino acid sequence consisting of amino acid residues 70 to 76 of SEQ ID NO: 10, and CDRL3 consisting of an amino acid sequence consisting of amino acid residues 109 to 117 of SEQ ID NO: 10,
more preferably an antibody comprising a heavy chain comprising a heavy chain variable region consisting of an amino acid sequence consisting of amino acid residues 20 to 142 of SEQ ID NO: 9 and a light chain comprising a light chain variable region consisting of an amino acid sequence consisting of amino acid residues 21 to 129 of SEQ ID NO: 10, and
even more preferably an antibody comprising a heavy chain consisting of an amino acid sequence consisting of amino acid residues 20 to 472 of SEQ ID NO: 9 and a light chain consisting of an amino acid sequence consisting of amino acid residues 21 to 234 of SEQ ID NO: 10, or a variant of the antibody in which a lysine residue at the carboxyl terminus of the heavy chain is deleted.

The average number of units of the drug-linker conjugated per antibody molecule in the anti-GPR20 antibody-drug conjugate is preferably 2 to 8, more preferably 3 to 8, even more preferably 7 to 8, even more preferably 7.5 to 8, and even more preferably about 8.

The anti-GPR20 antibody-drug conjugate can be produced with reference to descriptions in International Publication No. WO 2018/135501 and so on.

In the present invention, the term “anti-CDH6 antibody-drug conjugate” refers to an antibody-drug conjugate wherein the antibody in the antibody-drug conjugate according to the present invention is an anti-CDH6 antibody.

The anti-CDH6 antibody is preferably an antibody comprising a heavy chain comprising CDRH1 consisting of an amino acid sequence consisting of amino acid residues 45 to 54 of SEQ ID NO: 11, CDRH2 consisting of an amino acid sequence consisting of amino acid residues 69 to 78 of SEQ ID NO: 11, and CDRH3 consisting of an amino acid sequence consisting of amino acid residues 118 to 130 of SEQ ID NO: 11, and a light chain comprising CDRL1 consisting of an amino acid sequence consisting of amino acid residues 44 to 54 of SEQ ID NO: 12, CDRL2 consisting of an amino acid sequence consisting of amino acid residues 70 to 76 of SEQ ID NO: 12, and CDRL3 consisting of an amino acid sequence consisting of amino acid residues 109 to 116 of SEQ ID NO: 12,
more preferably an antibody comprising a heavy chain comprising a heavy chain variable region consisting of an amino acid sequence consisting of amino acid residues 20 to 141 of SEQ ID NO: 11 and a light chain comprising a light chain variable region consisting of an amino acid sequence consisting of amino acid residues 21 to 128 of SEQ ID NO: 12, and
even more preferably an antibody comprising a heavy chain consisting of an amino acid sequence consisting of amino acid residues 20 to 471 of SEQ ID NO: 11 and a light chain consisting of an amino acid sequence consisting of amino acid residues 21 to 233 of SEQ ID NO: 12, or a variant of the antibody in which the lysine residue at the carboxyl terminus of the heavy chain is deleted.

The average number of units of the drug-linker conjugated per antibody molecule in the anti-CDH6 antibody-drug conjugate is preferably 2 to 8, more preferably 3 to 8, even more preferably 7 to 8, even more preferably 7.5 to 8, and even more preferably about 8.

The anti-CDH6 antibody-drug conjugate can be produced with reference to descriptions in International Publication No. WO 2018/212136 and so on.

5. Therapeutic Agent and/or Method of Treatment

The therapeutic agent of the present invention comprises the antibody-drug conjugate used in the present invention. Further, the method of treatment of the present invention comprises administering the antibody-drug conjugate used in the present invention. The therapeutic agent and the method of treatment can be used for treatment of a metastatic brain tumor.

Primary cancers for the metastatic brain tumor for which the therapeutic agent and/or method of treatment of the present invention can be used are not particularly limited as long as they have a potential to metastasize to the brain, and breast cancer, lung cancer (including small cell lung cancer and non-small cell lung cancer), melanoma, renal cell cancer, kidney cancer, colorectal cancer (also called colon and rectal cancer, and including colon cancer and rectal cancer), gastric cancer (also called gastric adenocarcinoma), head and neck cancer, hepatocellular cancer, liver cancer, ovarian cancer, prostate cancer, bladder cancer, pancreatic cancer, endometrial cancer, thyroid cancer, malignant lymphoma and sarcoma can be exemplified, and breast cancer, lung cancer and melanoma can be preferably exemplified, breast cancer and lung cancer can be more preferably exemplified, and breast cancer can be even more preferably exemplified.

Among the antibody-drug conjugates used in the present invention, a preferable antibody-drug conjugate, particularly having a specific antibody, can be determined by examining the characteristics of the primary cancer and tumor markers. For example, in the case that the primary cancer is breast cancer or lung cancer and the expression of HER2 is found, an anti-HER2 antibody-drug conjugate can be preferably used; in the case that the expression of HER3 is found, an anti-HER3 antibody-drug conjugate can be preferably used; in the case that the expression of TROP2 is found, an anti-TROP2 antibody-drug conjugate can be preferably used; and in the case that the expression of B7-H3 is found, an anti-B7-H3 antibody-drug conjugate can be preferably used. Further, in the case that the primary cancer is melanoma and the expression of HER3 is found, an anti-HER3 antibody-drug conjugate can be preferably used; in the case that the expression of TROP2 is found, an anti-TROP2 antibody-drug conjugate can be preferably used; and in the case that the expression of B7-H3 is found, an anti-B7-H3 antibody-drug conjugate can be preferably used. Further, in the case that the primary cancer is kidney cancer and the expression of CDH6 is found, an anti-CDH6 antibody-drug conjugate can be preferably used.

The presence or absence of HER2, HER3, TROP2, B7-H3, GPR20 and CDH6, and other tumor markers, can be checked by, for example, collecting tumor tissues from a cancer patient and subjecting the formalin fixed paraffin embedded specimen (FFPE) to an examination at a gene product (protein) level, such as an immunohistochemistry (IHC) method, a flow cytometry, a western blot method, or an examination at a gene transcription level, such as an in situ hybridization method (ISH), a quantitative PCR method (q-PCR), or a microarray analysis; alternatively, it can also be checked by collecting cell-free blood circulating tumor DNA (ctDNA) from a cancer patient and subjecting to an examination which uses a method such as next-generation sequencing (NGS).

The therapeutic agent and method of treatment of the present invention can be preferably used for mammals, and can be more preferably used for humans.

The antitumor effect of the therapeutic agent and method of treatment of the present invention can be confirmed by, for example, generating a model in which cancer cells derived from a primary cancer into which a marker gene (for example, luciferase gene) has been transfected are transplanted into the brain of a test animal, and measuring, using any imaging technique, the life-prolonging effects and the transition of luminescence intensity of the marker when the therapeutic agent or method of treatment of the present invention is applied. For example, in the case of transplanting cells into which a luciferase gene has been transfected, luciferin is administered to investigate the luminescence intensity, and in the case that the luminescence intensity is diminished as compared with a control group when the therapeutic agent or method of treatment of the present invention is applied, an antitumor effect is recognized.

Alternatively, the antitumor effect of the therapeutic agent and method of treatment of the present invention can also be confirmed by generating a model in which a test animal is transplanted with a biopsy derived from a patient with a metastatic brain tumor (for example, a PDX brain transplant model) and applying the therapeutic agent or method of treatment of the present invention, and further can also be confirmed by administering the therapeutic agent or applying the method of treatment of the present invention to a patient with a metastatic brain tumor. The measurement of the antitumor effect can be carried out, for example, using CT, PET, and/or MRI, by confirming change in tumor volumes before and after applying the therapeutic agent or method of treatment of the present invention.

In addition, the antitumor effect of the therapeutic agent and method of treatment of the present invention can be confirmed, in a clinical study, with the Response Evaluation Criteria in Solid Tumors (RECIST) evaluation method, WHO'S evaluation method, Macdonald's evaluation method, measurement of body weight, and other methods; and can be determined by indicators such as Complete response (CR), Partial response (PR), Progressive disease (PD), Objective response rate (ORR), Duration of response (DoR), Progression-free survival (PFS), and Overall survival (OS).

The foregoing methods can provide confirmation of superiority in terms of the antitumor effect of the therapeutic agent and method of treatment of the present invention against metastatic brain tumors compared to existing anticancer agents.

The therapeutic agent and method of treatment of the present invention can show not only an antitumor effect against metastatic brain tumors, but also an antitumor effect against metastatic cancers other than metastatic brain tumors. Examples of the metastatic cancers other than metastatic brain tumors can include metastatic bone tumors, metastatic lung tumors and metastatic liver cancer, and metastatic bone tumors can be preferably exemplified. Metastatic cancers other than metastatic brain tumors may occur intercurrently with a metastatic brain tumor or may occur separately from a metastatic brain tumor, and the therapeutic agent and method of treatment of the present invention can exert an antitumor effect in both cases.

The therapeutic agent and method of treatment of the present invention can retard growth of cancer cells, suppress their proliferation, and further can kill cancer cells. These effects can allow cancer patients to be free from symptoms caused by cancer or can achieve an improvement in the QOL of cancer patients and attain a therapeutic effect by sustaining the lives of the cancer patients. Even if the therapeutic agent and method of treatment do not accomplish the killing of cancer cells, they can achieve higher QOL of cancer patients while achieving longer-term survival, by inhibiting or controlling the growth of cancer cells.

The therapeutic agent of the present invention can be expected to exert a therapeutic effect by application as systemic therapy to patients, and additionally, by local application to cancer tissues.

The therapeutic agent of the present invention can be administered as a pharmaceutical composition containing at least one pharmaceutically suitable ingredient. The pharmaceutically suitable ingredients can be appropriately selected and applied from formulation additives or the like that are generally used in the art, in view of the dosage, the administration concentration or the like of the antibody-drug conjugate used in the present invention. For example, the therapeutic agent of the present invention can be administered as a pharmaceutical composition (hereinafter, referred to as “the pharmaceutical composition of the present invention”) containing a buffer such as a histidine buffer, an excipient such as sucrose or trehalose, and a surfactant such as polysorbate 80 or 20. The pharmaceutical composition of the present invention can be preferably used as an injection, can be more preferably used as an aqueous injection or a lyophilized injection, and can be even more preferably used as a lyophilized injection.

In the case that the pharmaceutical composition of the present invention is an aqueous injection, it can be preferably diluted with a suitable diluent and then given as an intravenous infusion. For the diluent, a dextrose solution, physiological saline, and the like, can be exemplified, and a dextrose solution can be preferably exemplified, and a 5% dextrose solution can be more preferably exemplified.

In the case that the pharmaceutical composition of the present invention is a lyophilized injection, it can be preferably dissolved in water for injection, subsequently a required amount can be diluted with a suitable diluent and then given as an intravenous infusion. For the diluent, a dextrose solution, physiological saline, and the like, can be exemplified, and a dextrose solution can be preferably exemplified, and a 5% dextrose solution can be more preferably exemplified.

Examples of the administration route which may be used to administer the pharmaceutical composition of the present invention include intravenous, intradermal, subcutaneous, intramuscular and intraperitoneal routes, and preferably include an intravenous route. Further, the pharmaceutical composition of the present invention can also be administered by direct injection to the brain parenchyma, the pia mater or the arachnoid membrane.

The antibody-drug conjugate used in the present invention can be administered to a human once at intervals of 1 to 180 days, and can be preferably administered once a week, once every 2 weeks, once every 3 weeks or once every 4 weeks, and can be even more preferably administered once every 3 weeks. Also, the antibody-drug conjugate used in the present invention can be administered at a dose of about 0.001 to 100 mg/kg, and can be preferably administered at a dose of 0.8 to 12.4 mg/kg. In the case that the antibody-drug conjugate used in the present invention is an anti-HER2 antibody-drug conjugate, it can be preferably administered once every 3 weeks at a dose of 0.8 mg/kg, 1.6 mg/kg, 3.2 mg/kg, 5.4 mg/kg, 6.4 mg/kg, 7.4 mg/kg or 8 mg/kg, can be more preferably administered once every 3 weeks at a dose of 5.4, 6.4, or 7.4 mg/kg, and can be even more preferably administered once every 3 weeks at a dose of 5.4 mg/kg or 6.4 mg/kg. In the case that the antibody-drug conjugate used in the present invention is an anti-HER3 antibody-drug conjugate, it can be preferably administered once every 3 weeks at a dose of 1.6 mg/kg, 3.2 mg/kg, 4.8 mg/kg, 5.6 mg/kg, 6.4 mg/kg, 8.0 mg/kg, 9.6 mg/kg or 12.8 mg/kg, and can be more preferably administered once every 3 weeks at a dose of 4.8 mg/kg, 5.6 mg/kg or 6.4 mg/kg. In the case that the antibody-drug conjugate used in the present invention is an anti-TROP2 antibody-drug conjugate, it can be preferably administered once every 3 weeks at a dose of 0.27 mg/kg, 0.5 mg/kg, 1.0 mg/kg, 2.0 mg/kg, 4.0 mg/kg, 6.0 mg/kg or 8.0 mg/kg, and can be more preferably administered once every 3 weeks at a dose of 4.0 mg/kg, 6.0 mg/kg or 8.0 mg/kg.

The therapeutic agent of the present invention can also be administered in combination with a cancer therapeutic agent other than the antibody-drug conjugate used in the present invention, thereby enhancing the antitumor effect. Other cancer therapeutic agents used for such purpose may be administered to a subject simultaneously with, separately from, or subsequently to the therapeutic agent of the present invention, and may be administered while varying the administration interval for each. Such cancer therapeutic agents are not limited as long as they are agents having antitumor activity, and can be exemplified by at least one selected from the group consisting of irinotecan (CPT-11), cisplatin, carboplatin, oxaliplatin, fluorouracil (5-FU), gemcitabine, capecitabine, paclitaxel, docetaxel, doxorubicin, epirubicin, cyclophosphamide, mitomycin C, tegafur-gimeracil-oteracil combination, cetuximab, panitumumab, bevacizumab, ramucirumab, regorafenib, trifluridine-tipiracil combination, gefitinib, erlotinib, afatinib, methotrexate, pemetrexed, tamoxifen, toremifene, fulvestrant, leuprorelin, goserelin, letrozole, anastrozole, progesterone formulation, trastuzumab emtansine, trastuzumab, pertuzumab and lapatinib.

The therapeutic agent of the present invention can also be used in combination with radiotherapy. For example, a cancer patient may receive radiotherapy before and/or after receiving, or simultaneously with, the treatment by the therapeutic agent of the present invention. Examples of the radiotherapy method can include Whole brain radiation therapy (WBRT), Stereotactic irradiation (STI) and Stereotactic radiosurgery (SRS).

The therapeutic agent of the present invention can also be used as an adjuvant chemotherapy in combination with a surgical procedure. Surgical procedures are carried out by, for example, removing the whole or a part of a brain tumor. The therapeutic agent of the present invention may be administered for the purpose of diminishing the size of a brain tumor before a surgical procedure (referred to as pre-operative adjuvant chemotherapy, or neoadjuvant therapy), or may be administered after a surgical procedure for the purpose of preventing the recurrence of a brain tumor (referred to as post-operative adjuvant chemotherapy, or adjuvant therapy).

### EXAMPLES

The present invention is specifically described in view of the examples shown below. However, the present invention is not limited to these. Further, it is by no means to be interpreted in a limited way.

### Example 1: Production of Antibody-Drug Conjugate

In accordance with a production method described in International Publication No. WO 2015/115091 with use of a humanized anti-HER2 antibody (an antibody comprising a heavy chain consisting of an amino acid sequence consisting of amino acid residues 1 to 449 of SEQ ID NO: 1 and a light chain consisting of an amino acid sequence consisting of amino acid residues 1 to 214 of SEQ ID NO: 2), an antibody-drug conjugate in which a drug-linker represented by the following formula:

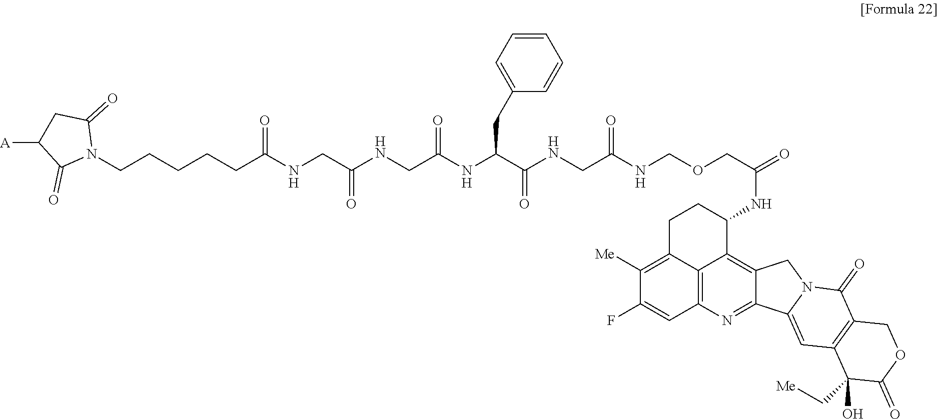

wherein A represents a connecting position to an antibody, is conjugated to the anti-HER2 antibody via a thioether bond (hereinafter, referred to as the “antibody-drug conjugate (1)”) was produced. DAR of the antibody-drug conjugate (1) is 7.8.

### Example 2: Life Prolongation Test

Mouse: 5 to 6-week-old female BALB/c nude mice (Charles River Laboratories Japan, Inc.) were subjected to the experiment.

The antibody-drug conjugate (1) was diluted with ABS buffer (10 mM acetate buffer (pH 5.5), 5% sorbitol) and intravenously administered into the tail vein at a solution volume of 10 mL/kg.

KPL-4-Luc was used, in which a luciferase gene was transfected into KPL-4 cells, a human breast cancer line obtained from Dr. Junichi Kurebayashi, Kawasaki Medical School (British Journal of Cancer, (1999) 79 (5/6). 707-717). KPL-4-Luc was suspended in physiological saline, 1×10⁴ cells were intracranially transplanted to female nude mice, and the mice were randomly grouped 7 days after the transplantation (Day 0). The antibody-drug conjugate (1) was intravenously administered at a dose of 10 mg/kg to the tail vein on Days 0 and 21. A solvent was administered to the control groups. The number of mice in each group was five and the presence or absence of survival was observed up to Day 28. From the viewpoint of animal ethics, in the case that a weight loss of 30% or more and abnormal behavior (circling movements, eating/drinking disorders, and the like) were found, euthanization was carried out. Further, luciferin was intraperitoneally administered to each mouse on Days 14, 21 and 28 to confirm tumor localization and amount of tumor with the luciferase activity of KPL-4-Luc as an indicator, and luminescence of the head was measured using In Vivo Imaging System (IVIS). A solvent-administered group (2 mice) was established as satellite, the formalin fixed paraffin embedded section of the mouse head was produced on Day 14, and hematoxylin and eosin staining was carried out.

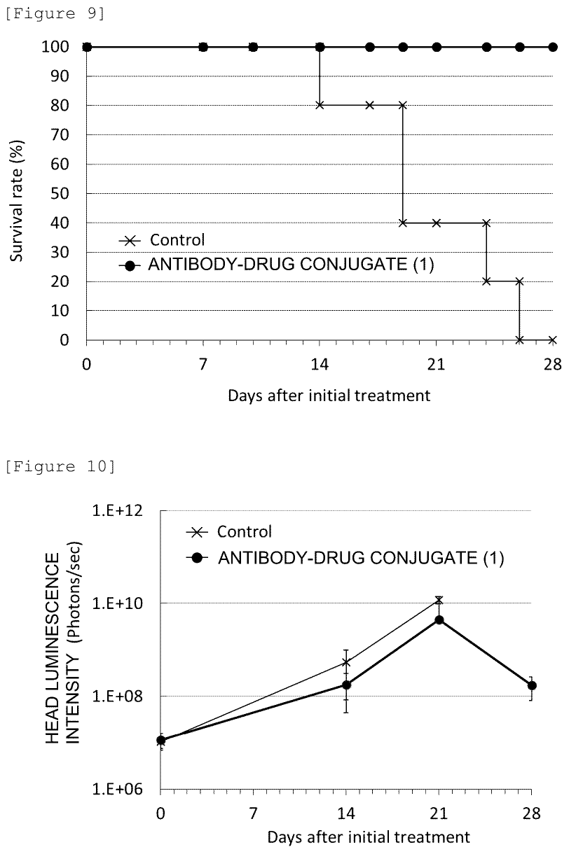

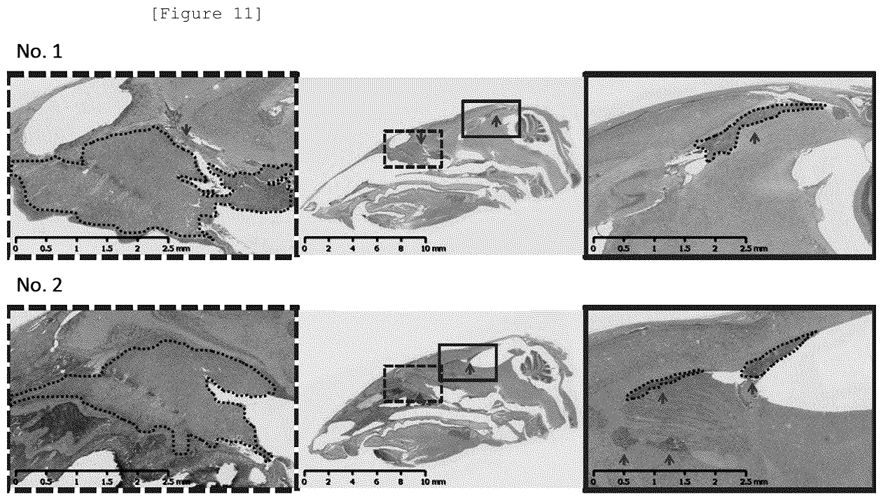

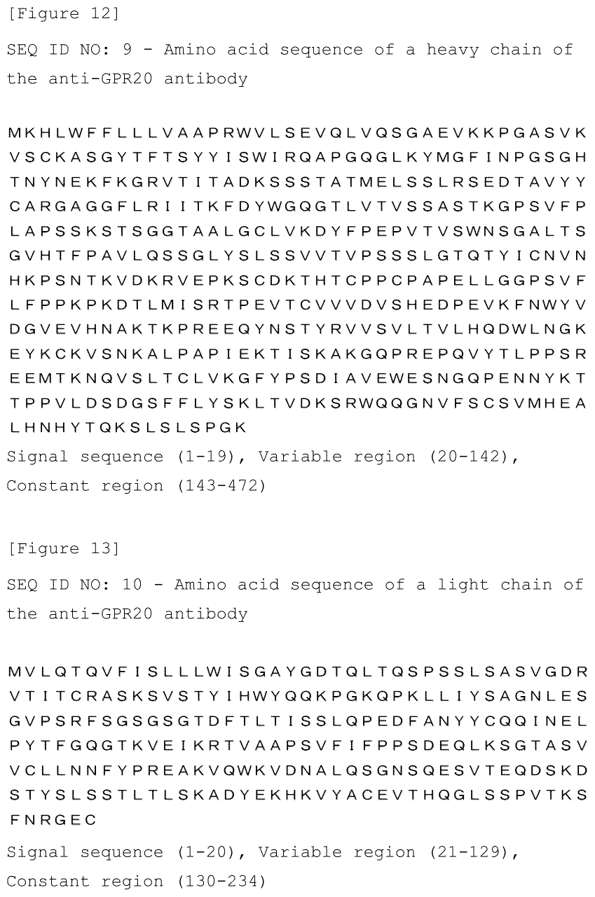

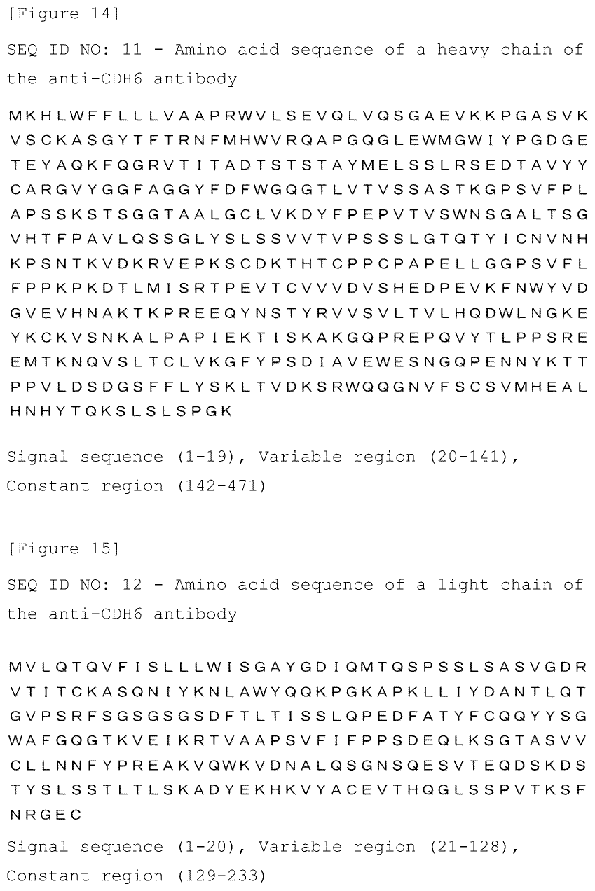

The results are shown in FIGS. 9 to 11. For the control groups, death was found from Day 14, and all of the mice died by Day 26. In contrast, for the antibody-drug conjugate (1)-administered groups, all the mice survived until Day 28 (FIG. 9). For the luminescence by KPL-4-Luc, the tumor growth was found in the mice of the antibody-drug conjugate (1)-administered groups by Day 21, but luminescence diminishment was found on Day 28, which was 1 week after the second administration, suggesting that the antibody-drug conjugate (1) exerts efficacy to tumors in the brain (FIG. 10). In the pathological images of the brain on Day 14, tumor masses were found around the olfactory bulb, in the nasal concha, the cerebral parenchyma, at the base of the brain, in the cerebral ventricle and around the cerebellum in 2/2 cases (FIG. 11).

These results suggested that the antibody-drug conjugate (1) exerts efficacy to metastatic brain tumors and exerts life-prolonging effects.

### Free Text of Sequence Listing

SEQ ID NO: 1—Amino acid sequence of a heavy chain of the anti-HER2 antibody
SEQ ID NO: 2—Amino acid sequence of a light chain of the anti-HER2 antibody
SEQ ID NO: 3—Amino acid sequence of a heavy chain of the anti-HER3 antibody
SEQ ID NO: 4—Amino acid sequence of a light chain of the anti-HER3 antibody
SEQ ID NO: 5—Amino acid sequence of a heavy chain of the anti-TROP2 antibody
SEQ ID NO: 6—Amino acid sequence of a light chain of the anti-TROP2 antibody
SEQ ID NO: 7—Amino acid sequence of a heavy chain of the anti-B7-H3 antibody
SEQ ID NO: 8—Amino acid sequence of a light chain of the anti-B7-H3 antibody
SEQ ID NO: 9—Amino acid sequence of a heavy chain of the anti-GPR20 antibody
SEQ ID NO: 10—Amino acid sequence of a light chain of the anti-GPR20 antibody
SEQ ID NO: 11—Amino acid sequence of a heavy chain of the anti-CDH6 antibody
SEQ ID NO: 12—Amino acid sequence of a light chain of the anti-CDH6 antibody

1.-62. (canceled)

63. A method of treating a metastatic brain tumor comprising, administering to a subject with a metastatic brain tumor an antibody-drug conjugate comprising a drug-linker conjugated to an antibody via a thioether bond, wherein the antibody-drug conjugate releases a drug having a structure of:

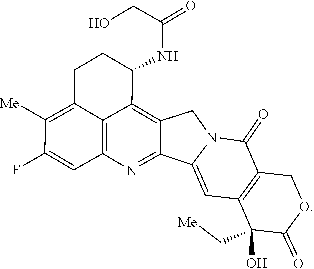 [SMILES: CC[C@@]1(O)C(=O)OCc2c1cc1n(c2=O)Cc2c-1nc1cc(F)c(C)c3c1c2[C@@H](NC(=O)CO)CC3]

64. The method of claim 63, wherein the metastatic brain tumor is metastasized from at least one primary cancer selected from the group consisting of breast cancer, lung cancer, melanoma, renal cell cancer, kidney cancer, colorectal cancer, gastric cancer, head and neck cancer, hepatocellular cancer, liver cancer, ovarian cancer, prostate cancer, bladder cancer, pancreatic cancer, endometrial cancer, thyroid cancer, malignant lymphoma, and sarcoma.

65. The method of claim 64, wherein the metastatic brain tumor is metastasized from at least one primary cancer selected from the group consisting of breast cancer, lung cancer, and ovarian cancer.

66. The method of claim 63, wherein the antibody is an anti-HER2 antibody, an anti-HER3 antibody, an anti-TROP2 antibody, an anti-B7-H3 antibody, an anti-GPR20 antibody, or an anti-CDH6 antibody.

67. The method according to claim 66, wherein the antibody is an anti-HER2 antibody.

68. The method according to claim 67, wherein the anti-HER2 antibody comprises a heavy chain consisting of amino acid residues 1 to 449 of SEQ ID NO: 1 and a light chain consisting of amino acid residues 1 to 214 of SEQ ID NO: 2.

69. The method according to claim 67, wherein the anti-HER2 antibody comprises a heavy chain consisting of the amino acid sequence of SEQ ID NO: 1 and a light chain consisting of the amino acid sequence of SEQ ID NO: 2.

70. The method according to claim 67, wherein the average number of units of the drug-linker conjugated per antibody molecule in the antibody-drug conjugate is in the range of from 2 to 8.

71. The method according to claim 66, wherein the antibody is an anti-HER3 antibody.

72. The method according to claim 71, wherein the anti-HER3 antibody comprises a heavy chain consisting of the amino acid sequence of SEQ ID NO: 3 and a light chain consisting of the amino acid sequence of SEQ ID NO: 4.

73. The method according to claim 71, wherein the anti-HER3 antibody comprises a heavy chain consisting of the amino acid sequence of SEQ ID NO: 3 in which a lysine residue at the carboxyl terminus of the heavy chain has been removed, and a light chain consisting of the amino acid sequence of SEQ ID NO: 4.

74. The method according to claim 71, wherein the average number of units of the drug-linker conjugated per antibody molecule in the antibody-drug conjugate is in the range of from 2 to 8.

75. The method according to claim 66, wherein the antibody is an anti-TROP2 antibody.

76. The method according to claim 75, wherein the anti-TROP2 antibody comprises a heavy chain consisting of amino acid residues 20 to 470 of SEQ ID NO: 5 and a light chain consisting of amino acid residues 21 to 234 of SEQ ID NO: 6.

77. The method according to claim 75, wherein the anti-TROP2 antibody comprises a heavy chain consisting of amino acid residues 20 to 469 of SEQ ID NO: 5 and a light chain consisting of amino acid residues 21 to 234 of SEQ ID NO: 6.

78. The method according to claim 75, wherein the average number of units of the drug-linker conjugated per antibody molecule in the antibody-drug conjugate is in the range of from 2 to 8.

79. The method according to claim 66, wherein the antibody is an anti-B7-H3 antibody.

80. The method according to claim 79, wherein the anti-B7-H3 antibody comprises a heavy chain consisting of amino acid residues 20 to 471 of SEQ ID NO: 7 and a light chain consisting of amino acid residues 21 to 233 of SEQ ID NO: 8.

81. The method according to claim 79, wherein the anti-B7-H3 antibody comprises a heavy chain consisting of amino acid residues 20 to 470 of SEQ ID NO: 7 and a light chain consisting of amino acid residues 21 to 233 of SEQ ID NO: 8.

82. The method according to claim 79, wherein the average number of units of the drug-linker conjugated per antibody molecule in the antibody-drug conjugate is in the range of from 2 to 8.

83. The method according to claim 66, wherein the antibody is an anti-GPR20 antibody.

84. The method according to claim 83, wherein the anti-GPR20 antibody comprises a heavy chain consisting of amino acid residues 20 to 472 of SEQ ID NO: 9 and a light chain consisting of amino acid residues 21 to 234 of SEQ ID NO: 10.

85. The method according to claim 83, wherein the anti-GPR20 antibody comprises a heavy chain consisting of amino acid residues 20 to 471 of SEQ ID NO: 9 and a light chain consisting of amino acid residues 21 to 234 of SEQ ID NO: 10.

86. The method according to claim 83, wherein the average number of units of the drug-linker conjugated per antibody molecule in the antibody-drug conjugate is in the range of from 2 to 8.

87. The method according to claim 66, wherein the antibody is an anti-CDH6 antibody.

88. The method according to claim 87, wherein the anti-CDH6 antibody comprises a heavy chain consisting of amino acid residues 20 to 471 of SEQ ID NO: 11 and a light chain consisting of amino acid residues 21 to 233 of SEQ ID NO: 12.

89. The method according to claim 87, wherein the anti-CDH6 antibody comprises a heavy chain consisting of amino acid residues 20 to 470 of SEQ ID NO: 11 and a light chain consisting of amino acid residues 21 to 233 of SEQ ID NO: 12.

90. The method according to claim 87, wherein the average number of units of the drug-linker conjugated per antibody molecule in the antibody-drug conjugate is in the range of from 2 to 8.

91. The method according to claim 63, wherein the antibody-drug conjugate releases the drug after the antibody-drug conjugate migrates into cancer cells of the metastatic brain tumor.

92. A method of treating a metastatic cancer comprising, administering to a subject with a metastatic cancer an antibody-drug conjugate comprising a drug-linker conjugated to an antibody via a thioether bond, wherein the antibody-drug conjugate releases a drug having a structure of:

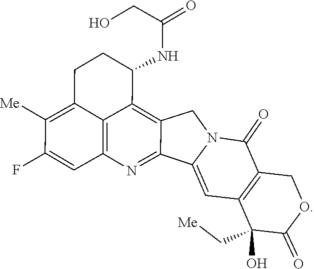 [SMILES: CC[C@@]1(O)C(=O)OCc2c1cc1n(c2=O)Cc2c-1nc1cc(F)c(C)c3c1c2[C@@H](NC(=O)CO)CC3]

93. A method of treating a metastatic brain tumor comprising administering to a subject with a metastatic brain tumor an antibody-drug conjugate, wherein the antibody-drug conjugate comprises a compound represented by the following formula conjugated to an antibody via a linker:

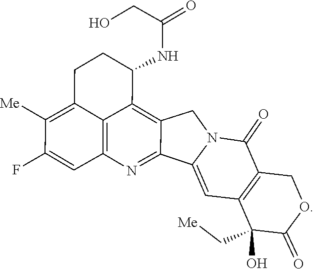 [SMILES: CC[C@@]1(O)C(=O)OCc2c1cc1n(c2=O)Cc2c-1nc1cc(F)c(C)c3c1c2[C@@H](NC(=O)CO)CC3]

94. The method of claim 93, wherein the compound is conjugated to the linker at the nitrogen atom of the amino group at position 1.

95. The method according to claim 93, wherein the linker is conjugated to the antibody via a thioether bond which is formed at an interchain disulfide bond site in the antibody.

96. The method according to claim 93, wherein the linker comprises a tetrapeptide residue having the formula Gly-Gly-Phe-Gly-.


SEQUENCE LISTING (12 sequences)

SEQ ID NO: 1 - protein, 450 aa, synthetic construct
  REGION 1..450: note = Heavy chain of anti-HER2 antibody
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, synthetic construct
  REGION 1..214: note = Light chain of anti-HER2 antibody
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 447 aa, synthetic construct
  REGION 1..447: note = Heavy chain of anti-HER3 antibody
QVQLQQWGAGLLKPSETLSLTCAVYGGSFSGYYWSWIRQPPGKGLEWIGEINHSGSTNYNPSLKSRVTISVETSKNQFSLKLSSVTAADTAVYYCARDKWTWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 220 aa, synthetic construct
  REGION 1..220: note = Light chain of anti-HER3 antibody
DIEMTQSPDSLAVSLGERATINCRSSQSVLYSSSNRNYLAWYQQNPGQPPKLLIYWASTRESGVPDRFSGSGSGTDFTLTISSLQAEDVAVYYCQQYYSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 470 aa, synthetic construct
  REGION 1..470: note = Heavy chain of anti-TROP2 antibody
MKHLWFFLLLVAAPRWVLSQVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 234 aa, synthetic construct
  REGION 1..234: note = Light chain of anti-TROP2 antibody
MVLQTQVFISLLLWISGAYGDIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 471 aa, synthetic construct
  REGION 1..471: note = Heavy chain of anti-B7-H3 antibody
MKHLWFFLLLVAAPRWVLSQVQLVQSGAEVKKPGSSVKVSCKASGYTFTNYVMHWVRQAPGQGLEWMGYINPYNDDVKYNEKFKGRVTITADESTSTAYMELSSLRSEDTAVYYCARWGYYGSPLYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 233 aa, synthetic construct
  REGION 1..233: note = Light chain of anti-B7-H3 antibody
MVLQTQVFISLLLWISGAYGEIVLTQSPATLSLSPGERATLSCRASSRLIYMHWYQQKPGQAPRPLIYATSNLASGIPARFSGSGSGTDFTLTISSLEPEDFAVYYCQQWNSNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 472 aa, synthetic construct
  REGION 1..472: note = Heavy chain of anti-GPR20 antibody
MKHLWFFLLLVAAPRWVLSEVQLVQSGAEVKKPGASVKVSCKASGYTFTSYYISWIRQAPGQGLKYMGFINPGSGHTNYNEKFKGRVTITADKSSSTATMELSSLRSEDTAVYYCARGAGGFLRIITKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 234 aa, synthetic construct
  REGION 1..234: note = Light chain of anti-GPR20 antibody
MVLQTQVFISLLLWISGAYGDTQLTQSPSSLSASVGDRVTITCRASKSVSTYIHWYQQKPGKQPKLLIYSAGNLESGVPSRFSGSGSGTDFTLTISSLQPEDFANYYCQQINELPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 471 aa, synthetic construct
  REGION 1..471: note = Heavy chain of anti-CDH6 antibody
MKHLWFFLLLVAAPRWVLSEVQLVQSGAEVKKPGASVKVSCKASGYTFTRNFMHWVRQAPGQGLEWMGWIYPGDGETEYAQKFQGRVTITADTSTSTAYMELSSLRSEDTAVYYCARGVYGGFAGGYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 233 aa, synthetic construct
  REGION 1..233: note = Light chain of anti-CDH6 antibody
MVLQTQVFISLLLWISGAYGDIQMTQSPSSLSASVGDRVTITCKASQNIYKNLAWYQQKPGKAPKLLIYDANTLQTGVPSRFSGSGSGSDFTLTISSLQPEDFATYFCQQYYSGWAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC